(19) United States
(12) Patent Application Publication
(10) Pub. No.: US 2010/0324150 A1
(43) Pub. Date: Dec. 23, 2010

(54) HYPERBRANCHED POLYGLYCEROL FOR IMPROVING HEART FUNCTION

(75) Inventors: Michael Allard, Vancouver (CA); Thomas J. Podor, Lions Bay (CA); Donald E. Brooks, Vancouver (CA); Rajesh K. Kainthan, Quincy, MA (US)

Correspondence Address: BLACK LOWE & GRAHAM, PLLC, 701 FIFTH AVENUE, SUITE 4800, SEATTLE, WA, 98104, US

(73) Assignee: THE UNIVERSITY OF BRITISH COLUMBIA, Vancouver, BC (CA)

(21) Appl. No.: 12/741,183

(22) PCT Filed: Oct. 31, 2008
(86) PCT No.: PCT/CA2008/001934

§ 371 Date: Aug. 2, 2010

Related U.S. Application Data
(60) Provisional application No. 60/996,137, filed on Nov. 2, 2007.

(51) Int. Cl. A61K 31/765; A61P 9/00

(52) U.S. Cl. 514/723

(57) ABSTRACT

A method of improving heart function in a subject, the method comprising administering an effective amount of a hyperbranched polyglycerol to a subject. The improvement in heart function may include one or more of an increase in myocardial contractile function, reduced or absent fibrosis, an increase in mechanical efficiency of the heart, an increase in ejection fraction, an increase in glucose oxidation or a decrease in fatty acid oxidation, as determined by conventional methods known in the art.

This application claims priority to U.S. Provisional Application 60/996,137 filed Nov. 2, 2007, which is herein incorporated by reference in its entirety.

The present invention relates to a method of improving heart function in a subject. The present invention also provides a method of improving heart function in a subject using hyperbranched polyglycerol.

### BACKGROUND OF THE INVENTION

Glucose and fatty acids are two sources of metabolic fuel used by the various tissues of the body. The preferred fuel under normal conditions varies with tissues, for example the brain to utilizes glucose almost exclusively, while a non-ischemic healthy heart may obtain ˜50-70% of the total energy from fatty acid oxidation, with the balance provided by glucose and other energy substrates.

In the tissues of the heart, particularly the myocardium, the availability of fatty acids is a key determinant of the rate of fatty acid oxidation in the heart. During surgical procedures for example, heart surgery, or when some pathological states are present for example, myocardial infarction, ischemic/reperfusion following an infarction, or diabetes mellitus, fatty acid concentrations are elevated, increasing fatty acid oxidation and decreasing glucose oxidation.

Some studies have suggested that myocardial contractile function may be improved by reducing fatty acid oxidation and shifting the metabolism to favour glucose oxidation (reviewed in Lopaschuk, 2006. Seminars in Cardiothoracic and Vascular Anesthesia 10:228-230). Patients receiving dichloroacetate (DCA) demonstrated increased glucose oxidation, and an increase in stroke volume, ejection fraction and an improvement in cardiac efficiency. Raising plasma insulin concentration also exhibited a similar effect of increasing glucose oxidation and reducing fatty acid oxidation.

Dichloroacetate has been shown to improve post ischemic function of hypertrophied hearts in a rat model, possibly by improving mechanical efficiency (Wambolt et al 2000. J Am College Cardiology 36:1378-1385).

Several agents are known that affect fatty acid oxidation in the heart and/or other tissues. Oxfenicine and etomoxir stimulate both glycolysis and glucose oxidation (reviewed in Lee et al 2004. Eur Heart J 25:634-641). PCT Publication WO 2006/125779 discloses that when extracellular glycerol concentration is increased, glycerol oxidation increased, whereas fatty acid beta-oxidation was reduced.

Subjects prescribed trimetazidine (a fatty acid oxidation inhibitor) also showed improvement in ejection fraction and contractility in over a six-month period (Rosano et al 2003. Cardoivasc Diabetol. 2:16).

Trimetazidine or ranolazine may shift cardiac energy metabolism from fatty acid oxidation to glucose oxidation (Kantor et al 2000. Circulation Research 86:580-588;). There have been links to Parkinsonism in some studies using trimetazidine (Marti Masso et al 2005. Therapie 60:419-422).

Insulin, in combination with glucose and potassium (GIK therapy) may lower circulating fatty acid concentration (Diaz et al 1998. Circulation 98:2227-2234)

Perhexiline inhibits a key enzyme in fatty acid metabolism in coronary tissues PCT Patent Application WO 05/087233 discloses a use of perhexiline for treatment of chronic heart failure.

Some beta-blockers have also been shown to decrease myocardial free fatty acid uptake (Wallhaus et al 2001. Circulation 103:2441-2446).

Various compounds have been disclosed as inhibitors of malonyl CoA decarboxylase inhibitors (PCT Patent Applications WO 2005/037258, WO 2005/011693 WO 2005/011670)

The above agents exert their metabolic effect by mimicking an enzyme substrate, for example, or by modulation of the function of one or more enzymes key to glucose or fatty acid metabolism. However, targeting of specific enzymes that are found in almost all tissues of the body may lead to toxicity concerns and secondary side effects An alternate approach to influence normal biochemical energy regulation towards a preferred energy substrate.

Various biocompatible polymers are known and have been used, or proposed for use, as drug delivery vehicles or carriers (see, for example, WO 2004/072153), or as hemoglobin substitute (WO 2005/052023). Other polymers for example linear or unbranched polyethylene glycols have been proposed for use as organ or tissue preservation (see, for example U.S. Pat. No. 6,949,335). A common general feature that makes such biocompatible polymers useful for in vivo applications is their lack of interaction, or minimal interaction with enzyme and tissues of the subject.

### SUMMARY OF THE INVENTION

The present invention relates to a method of improving heart function in a subject. The present invention also provides a method of improving heart function in a subject using hyperbranched polyglycerol.

The present invention further relates to methods of improving heart function in a subject comprising administering an effective amount of a hyperbranched polyglycerol to the subject.

In accordance with one aspect of the invention, there is provided a method of improving heart function in a subject, the method comprising administering an effective amount of a hyperbranched polyglycerol to a subject.

In accordance with another aspect of the invention, improving heart function comprises one or more of an increase in myocardial contractile function, reduced or absent fibrosis, an increase in mechanical efficiency of the heart, an increase in ejection fraction, an increase in glucose oxidation or a decrease in fatty acid oxidation.

In accordance with another aspect of the invention, there is provided a use of a hyperbranched polyglycerol for improving heart function in a subject.

In accordance with another aspect of the invention, there is provided a pharmaceutical composition comprising a hyperbranched polyglycerol and a pharmaceutically acceptable carrier in an amount effective to improve heart function.

In accordance with another aspect of the invention, the hyperbranched polyglycerol is alkylated.

In accordance with another aspect of the invention, the alkylated hyperbranched polyglycerol is selected from the group consisting of RKK-43, RKK-55, RKK-56, RKK-71, RKK-108, RKK-108′, RKK-108″, RKK-259, IC35, IC70 and IC40(1).

In accordance with another aspect of the invention, wherein the hyperbranched polyglycerol is non-alkylated.

In accordance with another aspect of the invention, the non-alkylated hyperbranched polyglycerol is selected from the group consisting of RKK-1, RKK-2, RKK-5, RKK-6, RKK-7, RKK-8, RKK-11, RKK-12, RKK-99, RKK-111, IC214 and IC72.

In accordance with another aspect of the invention, the amount effective to improve heart function is an amount that provides a concentration 0.001 μM to about 1000 μM, or any amount therebetween; from about 0.01 μM to about 1000 μM, or any amount therebetween; from about 0.1 μM to about 500 μM, or any amount therebetween; from about 1 μM to about 500 μM or any amount therebetween; from about 10 μM to about 400 μM or any amount therebetween; from about 20 μM to about 200 μM, or any amount therebetween; or from about 50 μM to about 200 μM or any amount therebetween.

In accordance with another aspect of the invention, an alkyl chain of the alkylated hyperbranched polyglycerol is a 4-carbon alkyl chain (C4), 5-carbon alkyl chain (C5), 6-carbon alkyl chain (C6), 7-carbon alkyl chain (C7), 8-carbon alkyl chain (C8), 9-carbon alkyl chain (C9), 10-carbon alkyl chain (C10), 11-carbon alkyl chain (C11), 12-carbon alkyl chain (C12), 13-carbon alkyl chain (C13), 14-carbon alkyl chain (C14), 15-carbon alkyl chain (C15), 16-carbon alkyl chain (C16), 17-carbon alkyl chain (C17), 18-carbon alkyl chain (C18), 19-carbon alkyl chain (C19) or a 20-carbon alkyl chain (C20).

In some aspects of the invention, the alkyl chain is a C18 or C10 group.

In accordance with another aspect of the invention, the effective amount provides a circulating blood concentration from about 20 μM to about 200 μM.

In accordance with another aspect of the invention, the hyperbranched polyglycerol has an average molecular weight of about 4 K to about 1200K or any amount therebetween; from 10K to about 750K or any amount therebetween; from about 20K to about 200K or any amount therebetween; from about 30K to about 100K or any amount therebetween; or from about 35K to about 90K or any amount therebetween, or any amount therebetween.

In accordance with another aspect of the invention, the hyperbranched polyglycerol has a mol % of glycidol endgroups from about 100% to about 50%, or any amount therebetween; from about 95% to about 55% or any amount therebetween; from about 90% to about 60% or any amount therebetween; from about 85% to abut 65% or any amount therebetween; or from about 80% to about 70%, or any amount therebetween.

In accordance with another aspect of the invention, the hyperbranched polyglycerol has a mol % of alkyl groups (R groups) from about 0% to about 15%, or any amount therebetween, from about 1% to about 14% or any amount therebetween; from about 2% to about 13%, or any amount therebetween; from about 3% to about 12%, or any amount therebetween; from about 4% to about 11% or any amount therebetween; from about 5% to about 10% or any amount therebetween; from about 6% to about 9% or any amount therebetween; or from about 7% to about 8% or any amount therebetween.

In accordance with another aspect of the invention, the hyperbranched polyglycerol has a mol % of PEG (polyethylene glycol or methoxypolyethylene glycol) comprising the hyperbranched polyglycerol polymers of the present invention may be from about 0% to about 35%, or any amount therebetween, from about 2% to about 34% or any amount therebetween; from about 4% to about 33%, or any amount therebetween; from about 6% to about 32%, or any amount therebetween; from about 8% to about 31% or any amount therebetween; from about 10% to about 30% or any amount therebetween; from about 12% to about 28% or any amount therebetween; from 14% to about 26%, or any amount therebetween, from about 16% to about 24% or any amount therebetween; or from about 18% to about 22%, or any amount therebetween.

In accordance with one aspect of the present invention, there is provided a method for modulating energy substrate use in a subject, the method comprising administering a composition comprising at least one species of hyperbranched polyether polyol to a subject. The subject may be diagnosed with, or suspected of having a cardiac disease or disorder.

The composition may comprise a hyperbranched polyether polyol at such a concentration and be administered in such a dose so as to provide a concentration in the blood of the subject in the range from about 0.001 uM to about 1000 uM.

In accordance with another aspect of the invention, there is provided a hyperbranched polyglycerol according to Formula 1:

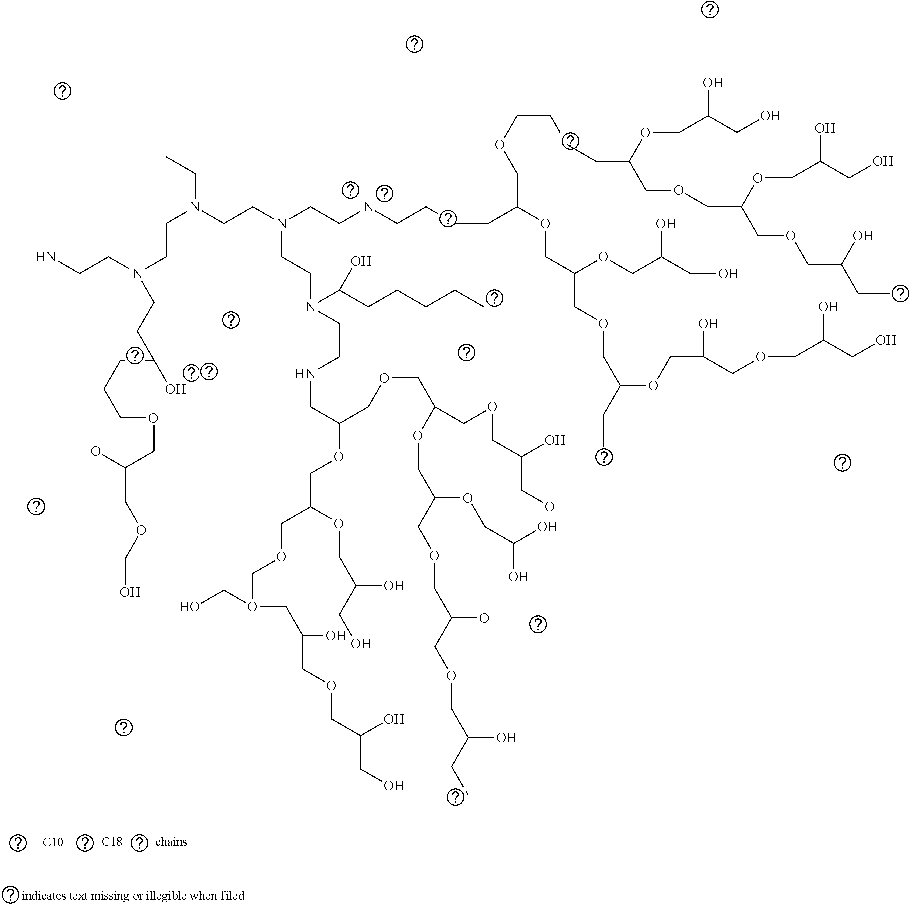

This summary of the invention does not necessarily describe all features of the invention. Other aspects, features and advantages of the present invention will become apparent to those of ordinary skill in the art upon review of the following description of specific embodiments of the invention.

### BRIEF DESCRIPTION OF THE DRAWINGS

These and other features of the invention will become more apparent from the following description in which reference is made to the appended drawings wherein:

FIG. 1 shows an effect of derivatized hyperbranched polyglycerol (dHPG) of the present invention on lactate production in H9C2 cells, relative to controls. (a) shows results of an initial assay of lactate production; (b) shows a subsequent repeat of the lactate production experiment, using an optimized normalization method (to cell protein). A bar graph depicting lactate concentration in H9C2 culture media at 6 hours following exposure to various concentrations of polymer (Polymer—IC35), 2.0 mM oxfenicene (OXF), 7.5 mM dichloroacetate (DCA) or 2.0 uM oligomycin (Oligo) is shown. Lactate concentration as % control is shown on the Y-axis. Data are Mean+/−SEM. *, vs Control, p<0.05.

FIG. 2 shows an effect of a dHPG of the present invention on heart function. Line plots of heart function in isolated working rat hearts (Beats per minute×mmHg/1000 on Y axis) over time (X axis) are shown. Control heart data is shown in open circles, RKK-108 (dHPG-85K-G_{71.4}-C18_{1.9}-PEG_{26.7}) treated heart data is shown with black circles. N=4 per group. *, different from Control, p<0.05.

FIG. 3 shows an effect of a dHPG of the present invention on substrate use in the intact heart. Bar graphs illustrate rates of palmitate oxidation, glucose oxidation and perfusate lactate levels in isolated rat hearts. Control data is shown in the white bars, RKK-108 (dHPG-85K-G_{71.4}-C18_{1.9}-PEG_{26.7}) treated heart data is shown in the black bars. N=4 per group. *, different from Control, p<0.05.

For all of FIGS. 4-18, dotted bar (far left), no infusion, n=5; striped bar (left of centre), Ringer's lactate only, n=2; white bar (centre), 10 uM RKK-108′ in Ringers's lactate; checked bar (right of centre), 1.2 mM RKK108′ in Ringer's lactate, n=3; black bar (far right), 1.2 mM RKK108′ in saline, n=3.

FIG. 4 shows a) the effect of RKK 108′ on blood pH (* P=0.27); b) the effect of RKK108′ on blood pCO₂(* P=0.049)

FIG. 5 shows a) the effect of RKK108′ on blood pO₂; b) the effect of RKK108′ on blood sO₂.

FIG. 6 a) the effect of RKK108′ on blood cBase(B)c; b) the effect of RKK108′ on blood cHCO₃(P)c.

FIG. 7 shows a) the effect of RKK108′ on ctHb (* P=0.025); b) the effect of RKK108′ on blood Hctc (* P=0.026).

FIG. 8 shows a) the effect of RKK 108′ on RBC count (* P=0.0163); b) the effect of RKK 108′ on blood haemoglobin (* P=0.154); c) the effect of RKK 108′ on % hematocrit in blood (* P=0.163).

FIG. 9 shows a) the effect of RKK 108′ on blood cNa+ (* P=0.035); b) the effect of RKK 108′ on blood cK+.

FIG. 10 shows a) the effect of RKK 108′ on blood cCl−; b) the effect of RKK 108′ on blood cCa²⁺ (* P=0.033).

FIG. 11 shows a) the effect of RKK 108′ on blood glucose (* P=0.034); b) the effect of RKK 108′ on blood cLactate.

FIG. 12 shows a) the effect of RKK 108′ on blood urea (* P=0.0485); b) the effect of RKK 108′ on blood creatinine.

FIG. 13 shows the effect of RKK 108′ on blood lactate dehydrogenase (LDH) (* P=0.037 for 1.2 mM RKK-108′ in saline, n=3) (* P=0.031 for 1.2 mM RKK-108′ in Ringers, n=2).

FIG. 14 shows a) the effect of RKK 108′ on blood aspartate amino transferase (AST); b) the effect of RKK 108′ on blood alanine amino transferase (ALT) (*P=0.014).

FIG. 15 shows a) the effect of RKK 108′ on blood white blood cell (WBC) count (*P=0.022); b) the effect of RKK 108′ on blood % neutrophils (*P=0.0015; **P=0.0194); c) the effect of RKK 108′ on blood % lymphocytes (*P=0.0016; **P=0.0358).

FIG. 16 shows a) the effect of RKK 108′ on % hemaotcrit in blood (*P=0.0163); b) the effect of RKK 108′ on blood mean corpuscular haemoglobin (MCH) (*P=0.01 for 1.2 mM RKK-108′ in saline, n=3) (*P=0.044 for 1.2 mM RKK-108′ in Ringers, n=2).

FIG. 17 shows a) the effect of RKK 108′ on blood mean corpuscular haemoglobin concentration (MCHC); b) the effect of RKK 108′ on blood RBC distribution width (RDW) (*P=0.046).

FIG. 18 shows a) the effect of RKK 108′ on blood platelet (PLT); b) the effect of RKK 108′ on blood mean platelet volume (MPV) (*P=0.026); the effect of RKK 108′ on blood platelet distribution width (PDW) (*P=0.029) (**P=0.025).

FIG. 19 shows the effect of C18 dHPG (IC35, dHPG-39K-G₇₈-C18_{1.6}-PEG₂₀) on substrate utilization (A) and recovery of function (B) during reperfusion after 24 min of no-flow global ischemia in isolated working rat hearts. Control, white bar. IC-35, black bar. N=6 to 18. Data represents a combination of studies using IC35 at concentrations of 20 and 50 μM. *, significantly different from Control, p<0.05

FIG. 20 shows the effect of dHPG on recovery of function during reperfusion of ischemic isolated working rat hearts. Control—open circles; IC35 treated hearts, solid circles.

FIG. 21 shows the effect of dHPG on post-ischemic function in vivo. Heart function was assessed non-invasively by echocardiography in hearts 5 days after a 30 min temporary coronary artery ligation in mice administered saline (Control; white bar) or C₁₈ dHPG (black bar) just prior to ischemia. Data was obtained in anesthetized mice prior to thoracotomy at day 0 and day 5. Values are expressed as % of pre-ischemic function. N=3 per group.

FIG. 22 shows the effect of dHPG on post-ischemic heart function in vivo. Representative in vivo left ventricular (LV) pressure signals (A,B) and LV pressure-volume (P-V) loops (C, D) 5 days after a 30 min temporary coronary artery ligation in mice treated with saline (Control) or IC35 just prior to ischemia. Bar graphs (E, F) show in vivo heart rate-LV pressure product 5 days after a 30 min temporary coronary artery ligation in mice treated with saline (Control) or C18 dHPG given just prior to ischemia (left) or upon reperfusion (right). N=2 to 3 per group. These measurements were obtained by means of a microtransducer introduced into the left ventricle via the apex of the left ventricle.

FIG. 23 shows an effect of different concentrations of dHPG according to the present invention on post-ischemic functional recovery of isolated working rat hearts. Control, open circle; IC-72, solid square; IC-35, solid circle; IC-214, open square.

FIG. 24 shows an effect of alkylated and non-alkylated dHPG of the present invention on substrate use in isolated working rat hearts after ischemia. Control, white bar; 20 micromolar IC-72, hatched bar; 20 micromolar IC-35 black bar.

FIG. 25 shows an effect of alkylated and non-alkylated dHPG of the present invention on substrate use in isolated working rat hearts after ischemia. Control, white bar; 50 micromolar IC-35, black bar.

### DETAILED DESCRIPTION

The present invention relates to a method of improving heart function in a subject. The present invention also provides a method of improving heart function in a subject using hyperbranched polyglycerol

The following description is of a preferred embodiment.

The present invention relates to a method of modulation of energy substrate use in a cell or tissues, of a subject.

The present invention further provides for the use of one or more hyperbranched polyether polyols for modulating modulation of the metabolism of a cell. As described herein, by administering one or more hyperbranched polyether polyols to a cell or cells, the energy substrate use of the cell or cells may be shifted from fatty acid oxidation to glucose oxidation. Furthermore, these polymers do not interact with a subject's enzyme or cellular systems.

Without wishing to be bound by theory, sequestration of exogenous fatty acids by hyperbranched polyether polyols provided by the present invention may reduce fatty acid oxidation in tissues or cells, with a corresponding, compensatory stimulation of glucose utilization. Stimulation of glucose utilization will be recognized by those skilled in the relevant art as beneficial to tissues or cells, in particular cells, or tissues of a subject.

The ability to modulate fatty acid oxidation in tissues or cells may be useful for maintaining or improving heart function following (or during) surgical procedures (for example open heart surgery, transplantation of an allograft heart or other organ, aortocoronary bypass grafting and the like), or in the presence of a pathological states such as a cardiac disease or disorder. When blood circulation is reduced or interrupted, the reduction, or absence of oxygen and nutrients that would normally be supplied by the circulating blood creates a condition in which the restoration of circulation induces oxidative stress and oxidative damage in the affected tissue or organ (for example a heart).

A variety of drugs are known to modulate fatty acid oxidation, but as discussed, undesirable side effects may arise when these drugs are administered systemically. Hyperbranched polyglycerol polymers of the present invention are useful for modulating fatty acid oxidation in tissue or cells, with the beneficial property that they do not adversely affect the tissues or cells of the subject.

The term “hyperbranched polyglycerol” as used in herein refers to a glycerol polymer having a plurality of branch points and multifunctional branches that lead to further branching with polymer growth. Hyperbranched polymers are obtained by a one-step polymerization process and form a polydisperse system with varying degrees of branching. Methods of making a variety of such polymers are known in the art (for example PCT/CA2006/000936), and further described herein.

The average molecular weight (Mn) of the hyperbranched polyglycerol polymers of the present invention may be from about 4 K to about 1200K, or any amount therebetween; from 10K to about 750K or any amount therebetween; from about 20K to about 200K or any amount therebetween; from about 30K to about 100K, or any amount therebetween; or from about 35K to about 90K, or any amount therebetween. For example, the average molecular weight of the hyperbranched polyglycerol polymers may be 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 32 0, 330, 340, 350, 400, 450, 500, 550, 600, 650, 700, 750, 800, 850, 900, 950, 1000, 1050, 1100, 1 150 or 1200 K, or any amount therebetween.

The mol % of glycidol endgroups comprising the hyperbranched polyglycerol polymers of the present invention may be from about 100% to about 50%, or any amount therebetween; from about 95% to about 55% or any amount therebetween; from about 90% to about 60% or any amount therebetween; from about 85% to abut 65% or any amount therebetween; or from about 80% to about 70% or any amount therebetween. For example, the mol % of glycidol end groups may be 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51 or 50 mol % or any amount therebetween.

The mol % of alkyl groups (R groups) comprising the hyperbranched polyglycerol polymers of the present invention may be from about 0% to about 15%, or any amount therebetween, from about 1% to about 14% or any amount therebetween; from about 2% to about 13%, or any amount therebetween; from about 3% to about 12%, or any amount therebetween; from about 4% to about 11% or any amount therebetween; from about 5% to about 10% or any amount therebetween; from about 6% to about 9% or any amount therebetween; or from about 7% to about 8% or any amount therebetween. For example, the mol % of alkyl groups (R-groups) may be 0, 1, 2, 3, 4, 5, 6, 7, 8, 9, 10, 11, 12, 13, 14 or 15 mol % or any amount therebetween.

The mol % of PEG (polyethylene glycol or methoxypolyethylene glycol) comprising the hyperbranched polyglycerol polymers of the present invention may be from about 0% to about 35%, or any amount therebetween, from about 2% to about 34% or any amount therebetween; from about 4% to about 33%, or any amount therebetween; from about 6% to about 32%, or any amount therebetween; from about 8% to about 31% or any amount therebetween; from about 10% to about 30% or any amount therebetween; from about 12% to about 28% or any amount therebetween; from 14% to about 26%, or any amount therebetween, from about 16% to about 24% or any amount therebetween; or from about 18% to about 22%, or any amount therebetween. For example the mol % of PEG may be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 mol % or any amount therebetween.

Selected hyperbranched polyglycerol polymers, with their mol % PEG and mol % R-groups, relative to the mol % glycidol endgroups are described in Table 1.

TABLE 1
Table of concordance for polymer code, polymer composition and experiment
designation.

| | Experiment | | | PEG | |
|---|---|---|---|---|---|
| Polymer code | designation | Glycidol | R mole % | mol % | Mn |
| HPG-4.2K-G₁₀₀-C18₀-PEG₀ | RKK-1 | 100 | 0 | 0 | 4200 |
| HPG-4.8K-G₁₀₀-C18₀-PEG₀ | RKK-2 | 100 | 0 | 0 | 4800 |
| HPG-4.6K-G₁₀₀-C18₀-PEG₀ | RKK-5 | 100 | 0 | 0 | 4600 |
| HPG-17.8K-G₁₀₀-C18₀-PEG₀ | RKK-6 | 100 | 0 | 0 | 17800 |
| HPG-36.2K-G₁₀₀-C18₀-PEG₀ | RKK-7 | 100 | 0 | 0 | 36200 |
| HPG-25.6K-G₁₀₀-C18₀-PEG₀ | RKK-8 | 100 | 0 | 0 | 25600 |
| HPG-140K-G₁₀₀-C18₀-PEG₀ | RKK-11 | 100 | 0 | 0 | 140000 |
| HPG-318K-G₁₀₀-C18₀-PEG₀ | RKK-12 | 100 | 0 | 0 | 318000 |
| HPG-44K-G₈₁-C18₂-PEG₁₇ | RKK-43 | 81 | 2 | 17 | 44000 |
| HPG-51K-G_{78.6}-C18_{1.4}-PEG₂₀ | RKK-55 | 78.6 | 1.4 | 20 | 51000 |
| HPG-51K-G_{70.7}-C18_{1.3}-PEG₂₈ | RKK-56 | 70.7 | 1.3 | 28 | 51000 |
| HPG-47K-G_{77.8}-C18_{1.2}-PEG₂₁ | RKK-71 | 77.8 | 1.2 | 21 | 47000 |
| dHPG-85K-G_{71.4}-C18_{1.9}-PEG_{26.7} | RKK-108 | 71.4 | 1.9 | 26.7 | 85000 |
| dHPG-36K-G₈₁-C18_{1.5}-PEG_{17.5} | RKK-108′ | 81 | 1.5 | 17.5 | 36000 |
| dHPG-37K-G_{74.9}-C18_{1.4}-PEG_{16.2}- | RKK-108″ | 74.9 | 1.4 | 16.2 | 37000 |
| (SO₃H)_{7.5} | | | | | |
| dHPG-80K-G₆₉-C18_{3.2}-PEG_{27.8} | RKK-153 | 69 | 3.2 | 27.8 | 80000 |
| dHPG-160K-G₅₆-C18_{2.5}-PEG₄₁ | RKK-148 | 56 | 2.5 | 41 | 160000 |
| dHPG-39K-G₇₃-C18₀-PEG₂₇ | RKK-99 | 73 | 0 | 27 | 39000 |
| HPG-1200K-G_{72.7}-C18_{4.3}-PEG₂₃ | RKK-52 | 72.7 | 4.3 | 23 | 1200000 |
| HPG-750K-G_{76.6}-C18_{2.4}-PEG₂₁ | RKK-112 | 76.6 | 2.4 | 21 | 750000 |
| HPG-100K-G₁₀₀-C18₀-PEG₀ | RKK-111 | 65 | 2 | 33 | 100000 |
| dHPG-180K-G_{68.5}-C10₁₃-PEG_{18.5} | RKK-259 | 68.5 | 13 | 18.5 | 180000 |
| dHPG-39K-G₇₈-C18_{1.6}-PEG₂₀ | IC35 | 78 | 1.6 | 20 | 39000 |
| dHPG-33K-G_{93.2}-C10_{3.4}-PEG₀-N_{3.4} | IC70 | 93.2 | 3.4 | 0 | 33000 |
| dHPG-35K-G₆₉-R₀-PEG₃₁ | IC72 | 69 | 0 | 31 | 35000 |
| dHPG-91K-G_{75.8}-C18_{2.2}-PEG_{18.2}-N_{3.8} | IC40(1) | 78 | 2.1 | 19.5 | 91000 |
| dHPG-36K-G₈₁-C18_{1.5}-PEG_{17.5} | IC6 | 81 | 1.5 | 17.5 | 36000 |
| dHPG-36.9K-G₇₅-R₀-PEG₂₅ | IC214 | 75 | 0 | 25 | 36900 |

Examples of R-groups include alkyl groups (for example C18, C10), or substituted alkyl groups. Information designating the HPG core may also be provided in the polymer code, as exemplified in the first column of Table 1. Table 1 lays out the average molecular weight (Mn), and the mol % PEG (PEG350) and mol % R-groups relative to the mol % glycidol for each of the polymers produced by the designated experiments and represented by the corresponding polymer code. Polymers of the present invention may be generally referred to by an experiment designation (for example RKK-1) for the sake of brevity, rather than the polymer code detailing the average molecular weight (Mₙ), and mol % PEG (PEG-350) and mol % R-groups, relative to the mol % glycidol, along with any additional derivative groups. As an example of this polymer code, experiment designation RKK-1 provides the polymer described by the polymer code HPG-4.2K-G₁₀₀-C18₀-PEG_{O}, which is an HPG polymer with an average molecular weight (Mₙ) of 42000, and 100 mol % of glycidol endgroups (no PEG or R-groups). As another example of this polymer code, the experiment designation IC40(1) provides the polymer described by the polymer code dHPG-91K-G_{75.8}-C18_{2.2}-PEG_{18.2}-N_{3.8}, which is a derivatized HPG polymer with an average molecular weight of 91000, 75.8 mol % glycidol, 2.2 mol % C18 alkyl R groups, 18.2 mol % PEG and 3.4 mol % amine groups (“N”) from an alkylated polyamine core used in preparation of the polymer. As another example of this polymer code, experiment designation RKK-108″ (“double prime”) provides the polymer described by the polymer code dHPG-37K-G_{74.9}-C18_{1.4}-PEG_{16.2}-(SO₃H)_{7.5}, and is a derivatized HPG polymer with an average molecular weight of 37000, 74.9 mol % glycidol, 1.4 mol % C₁₋₈ alkyl R groups, 16.2 mol % PEG and 7.5 mol % SO₃H groups

Hyperbranched polyglycerols of the present invention may alternately be described as alkylated, or non-alkylated. Examples of non-alkylated hyperbranched polyglycerols include RKK-1 (HPG-4.2K-G₁₀₀-C18₀-PEG₀), RKK-2(HPG-4.8K-G₁₀₀-C18₀-PEG₀), RKK-5 (HPG-4.6K-G₁₀₀-C18₀-PEG₀), RKK-6 (HPG-17.8K-G₁₀₀-C18₀-PEG₀), RKK-7 (HPG-36.2K-G₁₀₀-C18₀-PEG₀), RKK-8 (HPG-25.6K-G₁₀₀-C18₀-PEG₀), RKK-11 (HPG-140K-G₁₀₀-C18₀-PEG₀), RKK-12 (HPG-318K-G₁₀₀-C18₀-PEG₀), RKK-99 (dHPG-39K-G₇₃-C18₀-PEG₂₇), RKK-111 (HPG-100K-G₁₀₀-C18₀-PEG₀), IC214 (dHPG-36.9K-G₇₅-R₀-PEG₂₅) and IC72 (dHPG-35K-G₆₉-R₀-PEG₃₁). Examples of alkylated hyperbranched polyglycerols include RKK-43 (HPG-44K-G₈₁-C18₂-PEG₁₇), RKK-55 (HPG-51K-G_{78.6}-C18_{1.4}-PEG₂₀), RKK-56 (HPG-51K-G_{70.7}-C18_{1.3}-PEG₂₈), RKK-71 (HPG-47K-G_{77.8}-C18_{1.2}-PEG₂₁), RKK-108 (dHPG-85K-G_{71.4}-C18_{1.9}-PEG_{26.7}), RKK-108′ (dHPG-36K-G₈₁-C18_{1.5}-PEG_{17.5}), RKK-108″ (dHPG-37K-G_{74.9}-C18_{1.4}-PEG_{16.2}-(SO₃H)_{7.5}), RKK-259 (dHPG-180K-G_{68.5}-C10₁₃-PEG_{18.5}), IC35 (dHPG-39K-G₇₈-C18_{1.6}-PEG₂₀), IC70 (dHPG-33K-G_{93.2}-C10_{3.4}-PEG₀-N_{3.4}) and IC40(1) (dHPG-91K-G_{75.8}-C18_{2.2}-PEG_{18.2}-N_{3.8}).

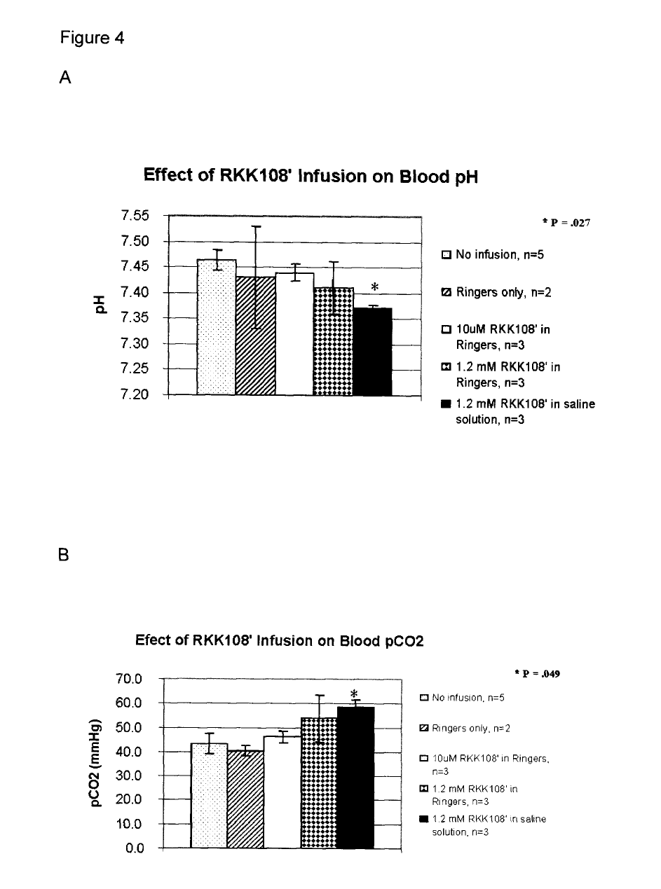

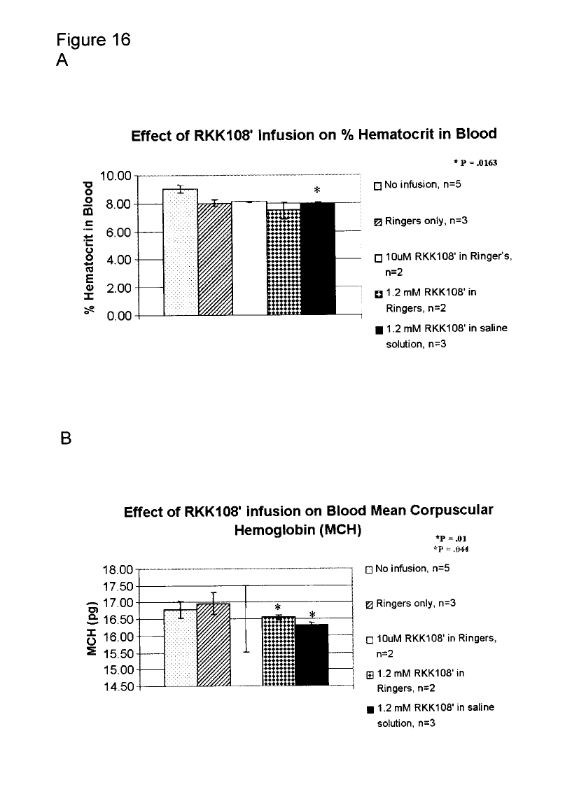

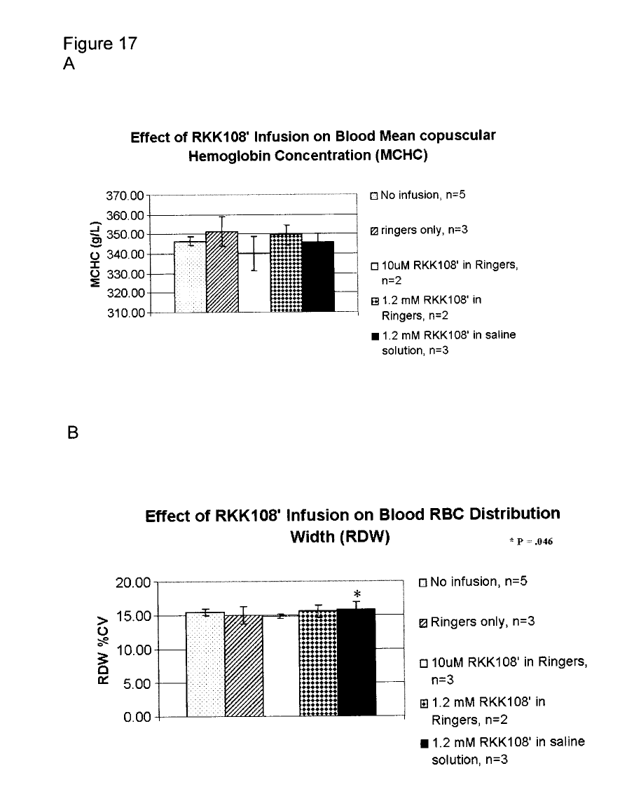

Hyperbranched polyglycerols (both alkylated and non-alkylated) are well-tolerated by mice, even when administered in high doses (Kainthan et al., 2006. Biomacromolecules 7:703-709; Kainthan et al, 2006. Biomaterials 27:5377-5390). No significant alteration of blood gases, blood cell numbers or function or induction of tissue indicators are observed (see Example 3, FIGS. 4-18).

One or more species hyperbranched polyglycerol polymers may be administered to a subject in an effective amount. An effective amount is an amount that achieves the intended effect for example modulation of metabolism of cells or tissues. An example of an effective amount of a hyperbranched polyglycerol is the quantity necessary to achieve a circulating blood concentration of about 0.001 μM (micromolar) to about 1000 μM (micromolar) or any amount therebetween, in a subject, or in the medium for maintaining an isolated organ or tissue (for example a cardiac allograft). The mass quantity of the hyperbranched polyglycerol necessary to achieve such a concentration will depend on the mass of the subject or volume of the medium, and calculation of such a quantity is within the ability of one skilled in the relevant art.

As examples, a hyperbranched polyglycerol may be provided to achieve a circulating blood concentration of about 0.001 μM to about 1000 μM, or any amount therebetween; or from about 0.01 μM to about 1000 μM, or any amount therebetween; or from about 0.1 μM to about 500 μM, or any amount therebetween; from about 1 μM to about 500 μM or any amount therebetween; from about 10 μM to about 400 μM or any amount therebetween; from about 20 μM to about 200 μM, or any amount therebetween, or from about 50 μM to about 200 μM or any amount therebetween. For example, the circulating blood concentration may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 205, 210, 215, 220, 225, 230, 235, 240, 24 5 or 250 μM (micromolar), or any amount therebetween.

As used herein, the term “modulation” includes up-regulation, induction, stimulation, potentiation, or relief of inhibition, as well as inhibition or down-regulation. Modulation may refer to an increase or decrease in a particular response or parameter, as determined by any of several assays generally known or used, some of which are exemplified herein. For example, the rate of glycolysis or glucose oxidation in a subject, or a tissue or organ of a subject, or heart function may be increased or improved, relative to a control by administration of one or more hyperbranched polyglycerol compounds to a subject.

A ‘subject’, as used herein, refers to a human patient or test subject, or a primate, or other mammal, such as a rat, mouse, dog, cat, cow, pig, sheep or the like.

Examples of tissues or organs include heart, liver, lung, spleen, kidney, skin, blood vessels, bone marrow and the like. In some examples, the organ is a heart, and the tissue is heart tissue. Cells may be specific to one particular tissue or organ, for example cardiac muscle cell, or may be found in multiple tissue or organs of a subject, for example fibroblasts, immune cells and the like. In some examples, the cell or tissue where modulation of energy substrate usage is, or is to, take place is capable of metabolizing glucose (or another sugar) and fatty acids as an energy substrate. Therefore, the invention provides for a method of modulation of energy substrate use, or reducing fatty acid oxidation, in a subject, or cell or tissue of a subject. The tissue, organ or cell may be in vivo, or ex vivo; in some examples the tissue, organ or cell may be in vitro (for example, a cell or tissue grown in culture, or an artificial organ grown in culture.)

Polymers (which may also be referred to as compounds) may be administered to a subject to alter the energy substrate usage systemically, or to alter the energy substrate usage of a tissue or organ. The one or more compounds may comprise a medicament (pharmaceutical composition) suitable for administration to a subject by any of several routes—the specific formulation of the medicament, including one or more pharmaceutically acceptable carriers or excipients, and quantity of the one or more compounds may vary depending on the route and the intended use.

A “pharmaceutically acceptable excipient” or carrier includes any and all solvents, dispersion media, coatings, antibacterial, antimicrobial or antifungal agents, isotonic and absorption delaying agents, and the like that are physiologically compatible. The excipient may be suitable for intravenous, intraarterial, intraperitoneal, intramuscular, intrathecal, intranasal, inhalation or oral administration. The excipient may include sterile aqueous solutions or dispersions for extemporaneous preparation of sterile injectable solutions or dispersion. Examples of sterile aqueous solutions include saline, Ringer's lactate or other solutions as may be known in the art. The choice of excipient will be dependent on the particular use or requirement to be met, for example, if the composition is to be injected, sterile Water for Injection may be a suitable excipient, whereas if the composition is to be administered orally, the excipient may comprise a suspending agent. Pharmaceutically acceptable excipients include, for example, an aqueous vehicle such as Water for Injection, Ringer's lactate, isotonic saline, salts, buffers, antioxidants, complexing agents, tonicity agents, cryoprotectants, lyoprotectants, suspending agents, emulsifying agents, antimicrobial agents, preservatives, chelating agents, binding agents, surfactants, wetting agents, non-aqueous vehicles such as fixed oils, waxes, creams or polymers or other agents for sustained or controlled release. See, for example, Berge et al. (1977. J. Pharm Sci. 66:1-19) or Remington—The Science and Practice of Pharmacy, 21st edition. Gennaro et al editors. Lippincott Williams & Wilkins Philadelphia, (both of which are herein incorporated by reference).

Routes of administration may be selected depending on the nature of the compound or composition to be delivered or the intended use. Examples of routes of administration include, for example, subcutaneous injection, direct injection into a disease site or tissue type, for example direct injection into a solid tumor, intraperitoneal injection, intramuscular injection, intravenous injection, epidermal or transdermal administration, mucosal membrane administration, ophthalmic, orally, nasally, rectally, topically, or vaginally. See, for example, Remington, The Science and Practice of Pharmacy, 21st edition. Gennaro et al. Editors. Lippincott Williams & Wilkins, Philadelphia. Carrier formulations may be selected or modified according to the route of administration. The amount of a pharmaceutical composition administered, where it is administered, the method of administration, the nature of the subject (for example age, gender, health status) and the timeframe over which it is administered may all contribute to the observed effect.

The compositions of the present invention may be formulated for administration by any of various routes. The medicaments may include an excipient in combination with an HPG polymer, and may be in the form of, for example, tablets, capsules, powders, granules, lozenges, pill, suppositories, aerosol, liquid or gel preparations. Medicaments may be formulated for parenteral administration in a sterile medium. The medicament may be dissolved or suspended in the medium. Compositions may be formulated for a subdermal implant in the form of a pellet, rod or granule. The implant or implants may be inserted subcutaneously by open surgery or by use of a trochar and cannula under local anaesthesia. The implant may be periodically replaced or removed altogether. Medicaments may also be formulated for transdermal administration using a patch. Specific methods, quantities, concentrations, excipients and compositions suitable for the various methods of administration will be known to one of skill in the art, and may be dependent on the desired use, or the condition of the subject.

As used herein, a “therapeutically effective amount” of a medicament, composition or compound refers to an amount of the medicament, composition or compound in such a concentration to result in a therapeutic level of drug delivered over the term that the drug is used. This may be dependent on mode of delivery, time period of the dosage, age, weight, general health, sex and diet of the subject receiving the medicament, composition or compound.

Compositions comprising a polymer according to various embodiments of the invention may be provided in a unit dosage form, or in a bulk form suitable for formulation or dilution at the point of use. Such compositions may be administered to a subject in a single-dose, or in several doses administered over time. Dosage schedules may be dependent on, for example, the subject's condition, age, gender, weight, route of administration, formulation, or general health. Dosage schedules may be calculated from measurements of adsorption, distribution, metabolism, excretion and toxicity in a subject, or may be extrapolated from measurements on an experimental animal, such as a rat or mouse, for use in a human subject. Optimization of dosage and treatment regimens are discussed in, for example, Goodman & Gilman's The Pharmacological Basis of Therapeutics 11ₜₕ edition. 2006. LL Brunton, editor. McGraw-Hill, New York, or Remington, The Science and Practice of Pharmacy, 21ₛₜ edition. Gennaro et al. Editors. Lippincott Williams & Wilkins, Philadelphia.

In some embodiments of the invention, compositions comprising at least one hyperbranched polyglycerol (HPG) polymer, or derivatized hyperbranched polyglycerol (dHPG) polymer may be administered to a subject exhibiting a cardiac disease or disorder. Examples of cardiac diseases or disorders include, but are not limited to, ischemia, acute cardiac ischemia and reperfusion, myocardial infarction, angina, hypertrophied heart, cardiac surgery, Type 1 diabetes mellitus, Type 2 diabetes mellitus, metabolic syndrome, acute or chronic heart failure, decreased contractile function, congestive heart failure, coronary artery graft surgery, cardioplegic arrest, ischemic cardiomyopathy, ischemic heart, pacing-induced heart failure, cardiopulmonary bypass surgery, diabetic cardiomyopathy, autoimmune disorders affecting the heart tissue, acidosis, and the like.

In some embodiments of the invention, a composition comprising one or more than one hyperbranched polyglycerol polymers may be administered to a subject exhibiting a cardiac disease or disorder. Without wishing to be bound by theory, administration of the hyperbranched polyglycerol polymer to the subject may improve heart function. The hyperbranched polyglycerol polymers may have different MW, different functional groups, different PEG group sizes, different alkyl chain groups and the like, as described herein and known in the art. Additionally, other agents may be co-administered with at least one hyperbranched polyglycerol polymers. Examples of such agents may include antioxidants, insulin or other hormones, chelating agents, pharmaceutical excipients, pharmaceutical agents that alter metabolism, alter oxidation and the like.

In some embodiments of the invention, hyperbranched polyglycerol polymers may be used in a medium or solution for preservation of an organ in anticipation of transplantation. An allograft organ, for example a heart may be perfused, or bathed with, a solution comprising one or more hyperbranched polyglycerol polymers before removal from the donor subject, or following removal from the donor subject.

In some embodiments of the invention, a composition comprising a hyperbranched polyglycerol may be used to systemically perfuse a donor subject providing an allograft organ for transplantation, before the organ is removed from the donor subject.

Therefore, the present invention also provides for a method useful for modulation of energy substrate use in a subject using hyperbranched polyglycerol, or a composition comprising hyperbranched polyglycerol. The present invention further provides for a method of improving heart function in a subject, or reducing fibrosis in a heart allograft using hyperbranched polyglycerol, or a composition comprising hyperbranched polyglycerol. An improvement in heart function may include, but is not limited to, an increase in myocardial contractile function, reduction or inhibition of fibrosis (which may be evidenced by an absence of fibrosis), an increase in mechanical efficiency of the heart, an increase in ejection fraction, an increase in glucose oxidation or a decrease in fatty acid oxidation.

The composition may be provided at an effective dose, such that the concentration of hyperbranched polyglycerol in the medium bathing an isolated organ or tissue, or the blood of the subject, or perfused into the tissue of the allograft is from about 0.001 μM to about 1000 μM, or any amount therebetween. The present invention further provides for use of an alkylated hyperbranched polyglycerol, present at a concentration of about 20 μM, about 50 μM or about 200 μM, for reducing fatty acid oxidation in heart tissue, or for increasing glucose oxidation in heart tissue or for improving heart function. For example, the hyperbranched polyglycerol may be present at a concentration of about 0.001 μM to about 1000 μM, or any amount therebetween; or from about 0.01 μM to about 1000 μM, or any amount therebetween; or from about 0.1 μM to about 500 μM, or any amount therebetween; from about 1 μM to about 500 μM or any amount therebetween; from about 10 μM to about 400 μM or any amount therebetween; from about 20 μM to about 200 μM, or any amount therebetween, or from about 50 μM to about 200 μM or any amount therebetween. For example, the circulating blood concentration may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 205, 210, 215, 220, 225, 230, 235, 240, 245 or 250 μM (micromolar), or any amount therebetween.

As a non-limiting example, the hyperbranched polyglycerol may be IC35, RKK-108, IC-72 or IC214, or a combination of one or more of these.

### Synthesis and Characterization of Hyperbranched Polyether Polyols

Methods are described in the art for the preparation of hyperbranched polyglycerol polymers (hyperbranched polyglycerols, or hyperbranched polyglycidols). Methods for making high molecular weight polyglycerol polymers, for example 20,000 and above are also known in the art. Methods of derivitazing or modifying such HPG to incorporate functional groups or other polymers into the polymer are known in the art. Derivatives of hyperbranched polymers may include polymers which contain hydrophobic and/or hydrophilic segments or portions which have been added to the polymer. Such portions may be provided by derivatization of terminal or branch hydroxyl groups on the hyperbranched polymer and/or by the addition of polymeric blocks to the branched polymer. Examples of such other polymers include, but are not limited to, poly(oxyalkylene) polymers, polyglycerol polymers, polyglycidol polymers, polyglycidol-block polymers, poly(glutamic acid) polymers, polyamidoamine (PAMAM) polymers, polyethyleneimine (PEI), polypropyleneimine (PPI) polymers, polymelamine polymers, polyester polymers, poly(lactic acid) polymers, epsilon poly(caprolactone), poly(lactone), substituted poly(lactones), poly(lactam), substituted poly(lactam), methoxy polyethylene glycol (MPEG or MePEG), polyethylene glycol (PEG), dextran, starch, cellulose, collagen, gelatine, chitosan and deacetylated chitosan. Examples of these methods may be found in, for example, Kautz et al 2001. Macromol Symp 163:67, Sunder et al. 1999 Macromolecules 32:4240, PCT/CA2006/000936, Kainthan R K and Brooks, D E. 2007 Biomaterials 28:4779-4787 and Sunder et al. 1999 Macromolecules 32:4240; Sunder et al. 2000. Chemistry 6 :2499-2506; Sunder et al. 1999. Angew Chem Int Ed Engl 3 :3552-3555; U.S. Pat. No. 6,822,068; PCT Patent Application WO 03/37532, Kainthan et al 2006. Macromolecules 39:7708-7717, all of which are incorporated herein by reference. An example of a synthetic scheme form hyperbranched polyglycerols is shown in Scheme 1.

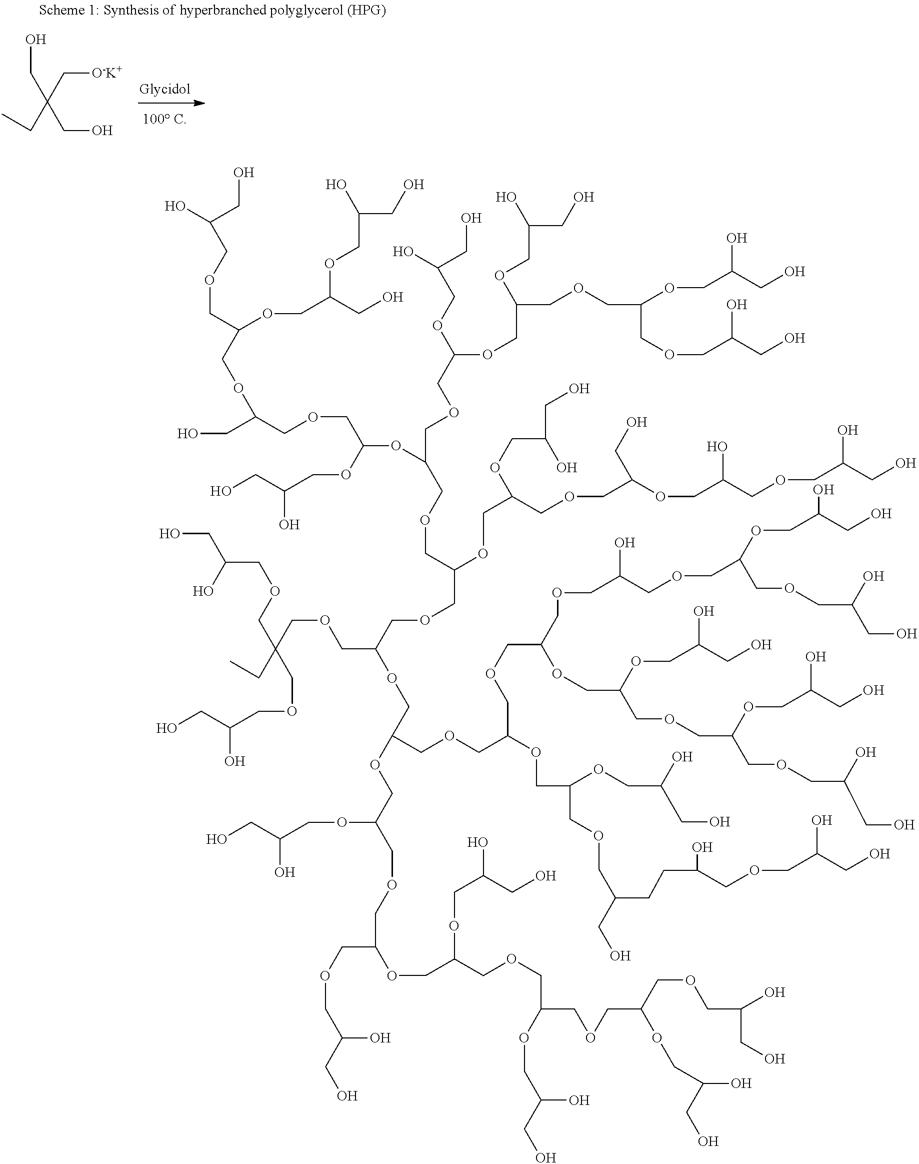

HPG polymers of the invention may be further derivatized (dHPG) with alkyl groups, polyethylene glycol groups, amine groups, sulfate groups and the like. An alkyl group refers to an organic sidechain comprising only hydrogen and carbon atoms arranged in a chain, having the general formula of CₙH₂ₙ₊₁. Alkyls may have primary, secondary, tertiary or quaternary substructure arrangements, depending on the carbon linking of the substituents. Use of such nomenclature is known in the art, for example IUPAC nomenclature of Organic Chemistry. For example, a primary alkyl having 3 carbons may be referred to as a “C3 alkyl group”. Other examples of alkyl groups include C4, C5, C6, C7, C8, C9, C10, C11, C12, C13, C14, C15, C16, C17, C18, C19 and C20. Exemplary methods described herein for the addition of alkyl groups to a hyperbranched polymer through an ether linkage may employ an epoxide precursor to provide at least one secondary hydroxyl group within the alkyl component added to the branched polymer.

A detailed polymerization procedure is described by Sunder et al. 1999 Macromolecules 32:4240, and modified as described in PCT/CA2006/000936 both of which are incorporated herein by reference. Following the polymerization reaction, the polymer was dissolved in methanol and neutralized by passing through a cation exchange resin (Amberlite IRC-150). The polymer was then precipitated into excess of acetone and stirred for 1 hour. Acetone was decanted out and this procedure repeated once. Polymers were dialyzed against water for 3 days, using cellulose acetate dialysis tubing (MWCO 1000 or 10,000 g/mol) with the water being changed three times per day. The polymer was lyophilized to dryness for future use.

If a higher molecular weight polymer is desired, a higher monomer/initiator core ratio may be employed but a greater polydispersity may also occur, along with increased viscosity. Kautz (Kautz et al 2001. Macromol Symp 163:67) describes a procedure to accommodate the viscosity of higher MW polymers. Use of alternate solvents for example diglyme (diethylene glycol dimethyl ether) as an emulsifying solvent, in combination with an increase in stirrer speed may also be helpful. Other solvents that may be used include THF or DMSO. See, for example, Kainthan R K and Brooks, D E. 2007 Biomaterials 28:4779-4787; Kainthan et al 2006 Macromolecules 39:7708-7717; and PCT/CA2006/000936, all of which are incorporated herein by reference.

A general synthetic scheme for HPGs with an alkylated polyamine core is disclosed in PCT/CA2006/000936, and Kainthan et al, 2006. Biomaterials 27:5377-5390, herein incorporated by reference.

An exemplary alkylated polyamine core for use in synthesis of some HPG polymers is shown in Scheme 2.

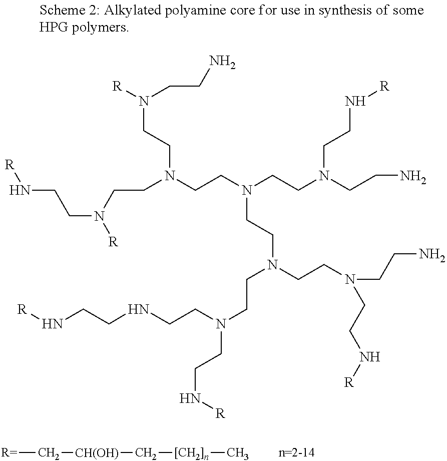

Scheme 2: Alkylated polyamine core for use in synthesis of some HPG polymers.

As an example, the HPG of Formula 1 exhibits a proportion of secondary amino groups that may be employed in further derivatization; a proportion of R-groups (in this example, they are C10 or C18 alkyl groups) and a plurality of hydroxyl groups (glycidol residues) that may be employed in further derivatization, for example, inclusion of other polymers (for example PEG or MePEG). One of skill in the art will, given the description and references provided herein, be able to manipulate the proportions and species of R-groups, polymers and secondary amines to obtain a particular combination, and verify the particular HPG composition or compositions obtained.

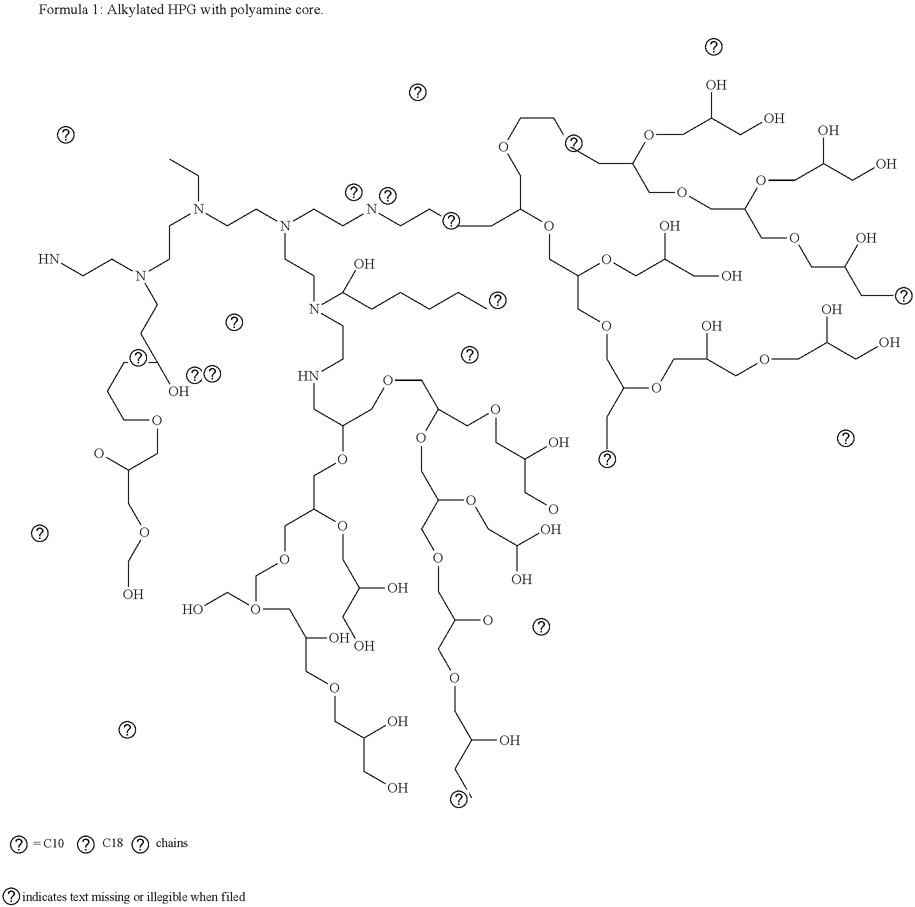

Modifications of this procedure utilizing dioxane as a dispersion media (as per references cited herein) provide a higher molecular weight HPG, with narrow molecular weight distributions.

A polymer comprising an —OH group at an end may be aminated following polymerization, using methods known in the art; for example, by dissolving in a polar aprotic solvent (for example pyridine, DMF, DMSO, diglyme or the like) and reacting with tosyl chloride. The resulting polymer-tosylate may be subsequently refluxed with an alkylamine, such as ethylamine in THF. Other solvents that may be used in this reflux include dioxane, DMF, DMSO, diglyme or the like.

Once synthesized, the HPG polymers may be characterized by NMR and gel permeation chromatography (GPC), using methods known in the art.

¹³C NMR provides gives information on both the degree of polymerization and the degree of branching (DB). The assignment of the peaks in ¹³C NMR spectra of HPGs has been well described (Sunder 1999 supra).

The molecular weight and polydispersity of the polymers may be obtained by GPC analysis. Use of both a Viscotek triple detector, which utilizes refractive index, 90-degree light scattering and intrinsic viscosity (N) determination and a multi-angle laser light scattering (MALLS) detector provides a measure of molecular weight distribution that does not rely on structural assumptions.

### Derivatization Chemistry

A single pot synthesis based on the epoxide ring opening reaction has been developed, and is described in PCT/CA2006/000936. Briefly, a synthetic methodology of this type avoids the formation of ester linkages, which may be susceptible to enzymatic hydrolysis by esterases. As a first step, an HPG of ˜7000-8000 g/mol may be synthesized using methods as described and referenced. In some syntheses, the epoxide of Brij 76 (decaethylene glycol octadecyl ether) may be used as a comonomer to provide a more reactive epoxide and avoid a purification step to get rid of unreacted monomer. Other comonomers may be used—quantitative conversions were obtained for phenyl glycidyl ether (PGE) and glycidyl 4-nonyl phenyl ether (GNPE) which were miscible with glycidol and were added as their mixtures.

Polymers containing sulfonic acid groups (for example RKK-108″) may be synthesized as described in PCT/CA2006/000936. Briefly, RKK-108 is dissolved in anhydrous THF and added to 100 mg of KH in a round-bottom flask containing 10 ml anhydrous THF. The mixture is stirred for abut 45 minutes, followed by addition of a solution of 1,3-propane sultone (30 mg), and stirring for an additional 12 hours. Solvent was evaporated and the polymer dissolved in water, the pH neutralized and purified by dialysis as described. The ratio of sulfonic acid groups may be varied by altering the amount of 1,3 propane sultone added.

Fatty acid binding: PCT/CA2006/000936 discloses the fatty acid binding properties of selected polymers. Fatty acid binding studies may be conducted by any of several methods known in the art—for example ¹³C NMR spectroscopy and titration calorimetry (Ugolini et al 2001. Eur J Biochem; Solowich et al 1997. Biochemistry 36:1719; Ragona et al 2000 Protein Science 9:1347).

Toxicological studies: All animal experiments were carried out under contract with the Advanced Therapeutics group at the B.C. Cancer Research Centre on the Vancouver Hospital site, as described in PCT/CA2006/000936; Kainthan et al, 2006. Biomaterials 27:5377-5390 and Kainthan et al., 2006. Biomacromolecules 7:703-709; all of which are incorporated herein by reference. No untoward indicators were found and all animals, even those with the very high doses of the highest molecular weight compound, grew normally with no signs of toxicity.

Pharmacokinetics: Pharmacokinetic analyses of selected polymers are described in PCT/CA2006/000936 and Kainthan et al, 2006. Biomaterials 27:5377-5390. RKK-43 and RKK-108 were assessed for circulation longevity and organ uptake.

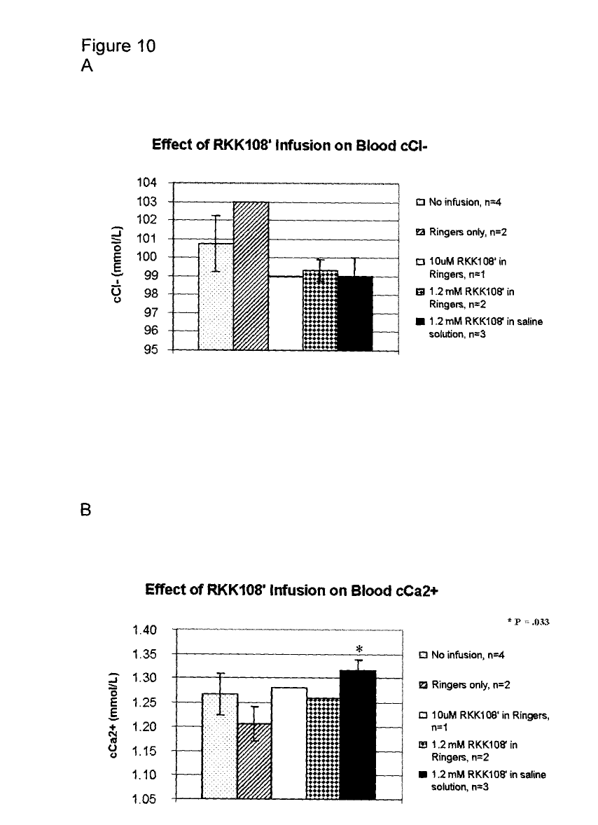

As previously described, RKK-43 was reported as being eliminated from the system faster than the higher molecular weight RKK-108. Diffusion from blood to tissues was reported as faster whereas the reverse process was slower compared to that of RKK-108. Organ and tissue retention of RKK-43 and RKK-108 polymers, and plasma half-life was also assessed. As reported in PCT/CA2006/00936, levels of RKK-43 and RKK-108 increased slowly in the spleens of the mice over the 30 days of experiment, with values ranging from 0.2 to 0.4 mg per gram tissue. The compound levels in lungs were very low but 0.1 and 0.2 mg/g tissue levels were observed on the 14th day. Constant levels of RKK-43 (0.1 mg/g) were found in the heart over the period of 30 days while it was found to increase slowly from 0.03 to 0.17 in the case of RKK-108. The highest tissue levels of RKK-43 and RKK-108 were observed in the livers, with levels being around 1.6 mg/g after two days. Levels of these polymers in the liver are shown as a function of time in FIG. 10. Higher amounts of low molecular weight RKK-43 containing 20% of PEG was accumulated in the liver compared to RKK-108 which contains 40% PEG. The plasma half-life of RKK-108 was found to be about 33 hr.

Coagulation studies: As described in PCT/CA2006/000936, Kainthan et al, 2006. Biomaterials 27:5377-5390 and Kainthan et al., 2006. Biomacromolecules 7:703-709, several polymers were tested for blood compatibility using the activated partial thromboplastin time (APTT) and the prothrombin time (PT) in fresh human plasma. RKK-28, comprising a polyglycerol core did not affect the coagulation pathways and the PT and APTT values were found to be similar to those of the controls. RKK-111, which is a copolymer of glycidol, epoxide of Brij-76 and PEG-epoxide increases PT and APTT considerably with increasing concentration. RKK-43 was found to increase the PT slightly and increase the APTT considerably. RKK-108 did not appear to have an effect on APTT or PT even at high concentration (10 wt %). RKK-153 which has higher alkyl content behaves similarly to RKK-43 (Bremerich et al 2000. Int. J Clin Pharmacol Therap. 38:408). MPEG (above a threshold value) may shield the PG and hydrophobic core from coagulation proteins.

Red cell aggregation and blood rheology: as described in PCT/CA2006/000936, Kainthan et al, 2006. Biomaterials 27:5377-5390 and Kainthan et al., 2006. Biomacromolecules 7:703-709, the response of red cells to RKK derivatives added to human blood in vitro was determined by microscopic examination and whole blood viscometry. Briefly, relative to control, the HPG core for example RKK-1 and RKK-108 had little to no effect on aggregation at 17 mg/ml. However, polymers with (a) lower MPEG content (RKK-43: 21%), (b) higher alkyl content (RKK-153; 4.5% octadecyl chains and 40% PEG chains (c) higher molecular weight MPEG chains (28% MPEG 550 cf MPEG 350 used for all other derivatives) and (d) a polymer containing Brij rather than C18 and MPEG chains, all demonstrated some degree of enhanced red cell aggregation.

Whole blood variable shear rate viscometry studies showed that RKK-43 (2.5% C18, 21% MPEG) and RKK-153 (4.5% C18, 40% MPEG) significantly elevated low shear rate viscosity while RKK-108 (2.6% C18, 38% MPEG) and RKK-28 which is polyglycidol core had little effect consistent with results of the aggregation studies.

As described in PCT/CA2006/000936, Kainthan et al, 2006. Biomaterials 27:5377-5390 and Kainthan et al., 2006. Biomacromolecules 7:703-709, complement activation was assessed. Studies were carried out in serum and plasma. Polymers RKK-108, RKK-153 and RKK-1 were assessed for complement activation relative to controls. Negligible complement activation was observed with the RKK polymers, relative to biocompatible polymers tested in parallel.

Platelet activation: as described in PCT/CA2006/000936, Kainthan et al, 2006. Biomaterials 27:5377-5390 and Kainthan et al., 2006. Biomacromolecules 7:703-709, platelet activation was assessed. RKK-28, RKK-108, RKK-153 and RKK-43 were compared to controls (PRG-350, Hetastarch, PVP, dextran and saline for platelet activation. All RKK compounds tested at up to 2 wt % caused ≦25%, platelet activation expression, suggesting that these compounds have little or no direct effect on platelets.

Plasma protein precipitation: as described in PCT/CA2006/00936, plasma protein precipitation was assessed. RKK-1, RKK-108, RKK-153 and PEG-350 at concentrations of 10 to 60 mg/ml in plasma did not exhibit visible flocculation or precipitation; the highest concentration employed in animal studies was only 10 mg/ml plasma.

### Model Systems and Assays for Assessing Modulation of Metabolism

An isolated, working rodent heart model is known in the art and an accepted model for metabolic drug discovery and development (Cheng et al, 2006. J Med Chem 2006 49:4055-58; Cheng et al, 2006. Bioorg Med Chem Lett 16:3484-88; Cheng 2006. J Med Chem 49:1517-25). Isolated, working hearts allow measurement of both function and metabolism, making it possible to assess efficiency of muscle performance from metabolic flux data as well as dose-response and structure-activity relationships of molecules in the intact heart.

Cultured heart muscle cells have also been used for studies of metabolism and in drug development. Such cells may be obtained from neonatal or adult rodents. Alternatively, H9C2 cells, a cell line derived embryonic rat heart ventricle, may be used as a model for cardiac cell metabolism.

While isolated rodent hearts provide a whole organ dataset, a cell culture model enables screening or testing of a greater number of samples.

When [U-¹⁴C]-glucose or [9,10-³H]-palmitate are catabolized in heart muscle cell mitochondria, ¹⁴CO₂ and ³H2O are released, respectively. Similarly, ³H₂O is released as [5-³H]-glucose is catabolized by glycolysis. Quantitative collection of ¹⁴CO₂ or ³H₂O produced by hearts can, therefore, be used to measure oxidation of glucose and palmitate and glycolysis in hearts. Alterations in the amount of lactate may be considered to be a reflection of underlying alterations in glucose use by the cells. Specifically, an increase in lactate will occur if rates of glycolysis are elevated with or without an increase in glucose oxidation. Since glycolytic rates exceed glucose oxidation rates, an increase in glycolysis will lead to enhanced lactate production, even if glucose oxidation is also stimulated. A decrease in lactate will occur if glucose oxidation is stimulated, leading to a greater utilization of pyruvate produced from glycolysis.

Various methods described herein may be used to assess cardiac function and/or metabolism of fatty acids, glucose and other energy substrates used by cardiac cells or tissue in culture, isolated rat heard models or in vivo studies. Other assays and methods that may also be used to assess cardiac function and/or metabolism of various energy substrates are known to those skilled in the art. Examples of such methods may include the following:

Myocardial Substrate Utilization Rates

Oxidation of palmitate and glucose may be assessed by quantitative collection of ¹⁴CO₂ released from labeled palmitate or glucose (¹⁴C or ³H) as a gas and dissolved in the perfusate as [¹⁴C]-bicarbonate (Allard, supra; Longnus et al 2001. Am J Physiol 281:H1561-H1567). Rates of glycolysis or palmitate oxidation may be determined by quantitatively measuring the rate of ³H₂O released into the perfusate from [5-³H]-glucose or [9,10-³H]-palmitate, respectively (Allard, supra; Lopaschuk, 1997 supra).

Myocardial Metabolites and PDH Activity

Adenine nucleotides and creatine phosphate may be determined in perchloric acid extracts of frozen ventricular tissue by high performance liquid chromatography in order to assess the energy status of the heart (Longnus et al 2003. Am J Physiol Regul Integr Comp Physiol 284:R936-44). Myocardial glycogen content may be determined following extraction from frozen ventricular tissue with 30% KOH, ethanol precipitation, and acid hydrolysis of glycogen (Henning et al 1996. Circulation 93:1549-1555). Total lipids may be extracted from frozen ventricular tissue following a chloroform/methanol extraction (Carr et al 1993. Clin Biochem 26:39-42). Triglyceride content may be determined using a colorimetric method (Roche Hitachi, Indianapolis, Ind., USA). Pyruvate dehydrogenase (PDH) activity, a major factor controlling oxidation of glucose in hearts, may be determined in homogenates of frozen ventricular tissue to determine if dHPG induced changes in its activity are responsible for any changes in glucose oxidation observed (Lydell et al 2002. 53:841-51).

### EXPERIMENTAL PROTOCOLS

Culture and Treatment of H9C2 cells

H9C2 (2-1) embryonic rat heart cells (passage 12, obtained from American Type Culture Collection, Manassas, Va.) were cultured in Dulbecco's modified Eagle's medium (DMEM—Gibco-Invitrogen) containing 10% fetal bovine serum (FBS) and 100 U/ml penicillin-streptomycin at 37° C. in a humidified atmosphere containing 5% CO₂. The cells were subcultured into 60 mm culture dishes when 80% confluent (before fusion into myotubes occurred) and were differentiated toward a cardiac phenotype by exposure to DMEM containing 1% horse serum and 0.1 μM all-trans retinoic acid (Sigma) for four days (Menard et al 1999. J Biol Chem 274:29063-70; Bronstrom et al 2000. Int J. Biochem Cell Biol 32:993-1006). Retinoic acid was prepared in the dark in DMSO and stored at −20° C. The concentration of DMSO in the culture media was less than 0.2%. Media was changed daily.

To screen for metabolic actions of dHPGs, H9C2 cells were exposed to a range of dHPG concentration (0.001 to 100 μM). Studies were conducted over six hours in serum-free Krebs-Henseleit (KH) solution (118 mM NaCl, 4.7 mM KCl, 1.2 mM KH₂PO₄, 1.2 mM MgSO₄, 2.0 mM CaCl₂, 25 mM NaHCO₃, 0.4 mM Na₂CO₃) containing 0.4 mM palmitate (bound to fatty-acid free albumin), 5.5 mM glucose and 20 mU/L insulin. Aliquots of media (60 μl) were taken at 3 and 6 hours. Viability of cells was assessed by visual inspection of cell morphology using a microscope and by assessment of mitochondrial integrity (MTS—Promega). Accumulation of lactate in the bathing solution over 6 hrs was measured using a diagnostic kit (Sigma, St. Louis, Mo.). Lactate accumulation was expressed as μmol/mg protein. Protein content of the cultures was determined using a commercial Bicinchoninic Acid (BCA) protein assay kit from Sigma (St. Louis, Mo.). Pharmacological agents used as controls included Dicholoracetate (DCA), which directly stimulates glucose oxidation by activating pyruvate dehydrogenase and causes a reduction in lactate. Oxfenicine (OXF) is an inhibitor of fatty acid transport into mitochondria, and causes an increase in lactate accumulation because of stimulatory effects on both glycolysis and glucose oxidation. Oligomycin (oligo) inhibits oxidative phosphorylation in mitochondria, and causes a large increase in lactate as a result of accelerated glycolysis.

Isolated Heart Preparation and Perfusion Protocol

Hearts from halothane (3-4%)-anesthetized male Sprague-Dawley rats were isolated and perfused as working preparations with Krebs-Henseleit (KH) solution at a left atrial preload of 11.5 mmHg and an aortic afterload of 80 mmHg in a closed recirculating system with oxygenated (95% O₂-5% CO₂) Krebs-Henseleit (KH) solution maintained at 37° C., as described (Burelle et al 2004. Am J Physiol Heart Circ Physiol 287:H1055-63; Allard et al 1994. Am J Physiol 267:H742-H750; Saeedi et al 2006. BMC Cardiovasc Disord 6:8). A first series of hearts was perfused with KH solution containing 0.6 or 1.2 mM [1-¹⁴C]- or [9,10-³H]-palmitate, prebound to fatty acid-free albumin (3%), together with 5.5 mM glucose, 0.5 mM lactate, and 20 mU/l insulin in order to measure rates of palmitate oxidation. A second series of hearts was perfused with KH solution containing 0.6 or 1.2 mM palmitate, 5.5 mM [5-³H/U-¹⁴C]-glucose, 0.5 mM lactate, and 20 mU/l insulin in order to measure glycolysis and glucose oxidation. Concentrations of insulin and substrates reflect values seen under physiologic and pathophysiologic conditions. The high palmitate concentration is used to recapitulate those seen during myocardial ischemia.

Heart rate and systolic pressure may be measured using a pressure transducer (Viggo-Spectramed, Oxnard, Calif.) inserted in the afterload line of the isolated heart. Cardiac output and aortic flow may be measured via external flow probes (Transonic Systems, Ithaca, N.Y.) on the left atrial preload and aortic afterload lines, respectively. External work performed by the heart is expressed as, “rate-pressure product”, the product of heart rate and peak systolic pressure, and “hydraulic work”, the product of cardiac output and peak systolic pressure. Perfusate and gas samples are taken every 10 min of non-ischemic perfusion and at 5, 10, 20, 30, and 40 minutes of reperfusion after ischemia. Hearts were frozen in liquid nitrogen at the end of perfusion for further analysis.

Isovolemic Blood Exchange Studies

Lewis rats (325-360 g weight) were anaesthesized using 4% isofluorane and maintained at 0.5%-2% during the surgical observation period of 3 hours. The O₂ saturation values and the heart rate (HR) were continuously monitored using an oxymeter (Nonin) clipped on to one of the animals paws. Catheters made of polyethylene tubing (Clay Adams PE 50) were inserted into each of the femoral artery and vein and were held in place with #5 silk sutures. The heparinized (30 UI/mL) femoral artery catheter was hooked up to a pressure transducer (AD Instrument) through a stopcock. The pulse pressure (PP) and HR were obtained at the start of blood exchange, periodically after the early part of the exchange, at 1.5 hours into the blood exchange, and at the end of blood exchange (approx. 3 hours post-infusion).

For the control animals (for pre-exchange blood status), blood was sampled immediately after cannulation from the femoral artery catheter: 150 uL for complete blood count (CBC); 300 uL for blood gases; 600 uL for kidney/liver function tests. The blood samples for blood gases and kidney/liver function tests were tested immediately following sampling, using standard clinical methods. The CBC analysis was conducted within 10 minutes of blood sampling, using standard clinical methods. The animals were euthanized after blood collection. Organ tissues (liver, kidney, spleen, pancreas, skeletal muscle and heart) were collected for future immunohistochemical analysis.

For the experimental animals (n=5), after obtaining PP and HR readings, the respective catheters were connected to the push-pull pump (Harvard Apparatus) where the femoral vein catheter was connected to the push 5-mL syringe (BD) while the arterial catheter was connected to the pull 5-mL syringe (via 23 gauge needles. The 15% TVE isovolemic exchange was performed at 200 uL/min. Control animals were treated with lactated Ringer's solution, experimental animals were treated with 10 uM or 1.2 mM dHPG-36K-G₈₁-C18_{1.5}-PEG_{17.5} (MW 83000) in lactated Ringer's solution. After the exchange, the animals while under maintenance anesthesia, were observed for ˜3 hours. At the end of 3 hours, blood samples were handled the same manner as in the control animal mentioned above. Organ tissue samples were also collected as above. % TVE was calculated according to weight=%×58 ml/kg weight of animal.

Blood pH, pCO₂ (CO₂ partial pressure), cHCO₃ (plasma bicarbonate concentration), cBASE(B)c (base excess), sO₂ (arterial oxygen saturation), ctHb (total hemoglobin concentration in blood), Hctc (capillary hematocrit), LDH (lactate dehydrogenase), cNa⁺ (plasma Na concentration), cK⁺ (plasma K concentration), cCa⁺² (plasma Ca concentration), cGlucose (plasma glucose concentration), cLactate (plasma lactate concentration), urea, creatinine, ALT (alanine aminotransferase), and AST (aspartate aminotransferase) analyses were performed using standard clinical methods at St. Paul's Hospital Clinical Laboratory (Vancouver BC). Blood count analyses (white blood cell (WBC), % neutrophils, % lymphocytes, red blood cell (RBC) count, hemoglobin, % hematocrit, mean corpuscular volume (MCV), mean corpuscular hemoglobin (MCH), mean corpuscular hemoglobin concentration (MCHC), RBC distribution width (RDW), platelet (PLT), mean platelet volume (MPV) and blood platelet distribution width (PDW) were performed using standard clinical methods (iCAPTURE Centre).

Polymers Used in H9C2 and Isolated Rat Heart Experiments

A subset of the polymers initially synthesized was selected for further investigation (Table 2) in H9C2 cells, isolated rat hearts and in isovolemic blood exchange studies.

Heart transplant models: With Lewis-to-Fisher 344 allografts and Lewis-to-Lewis isografts, the native heart of the rat receiving the transplant serves as an internal control for the systemic immune environment. Syngrafts were transplanted with donor hearts as described (Adams et al 1992, Transplantation. 53:1115-1119, 1992; Ono et al 1969. J Thorac Cardiovasc Surg. 57:225-229) that had been preserved for an estimated 20 min to 20 hours by perfusion with different doses of RKK108 in the same preservative used for the tissues. Animal experiments were approved by the University of British Columbia Committee on Animal in accordance with the Canadian Council on Animal Care. Rats were acclimatized for 1 week and weighed 200 to 225 g at the time of surgery. Histopathology of RKK108-perfused transplants with those of other preservative treated hearts.

Morphometry and Histochemistry Analysis: The extent of pathological changes and particularly the degree of rejection and transplant vasculopathy in the perfused heart tissue was calculated in paraformaldehyde (4%) perfusion-fixed hearts, sectioned into 3-5 one-mm-thick mid ventricular blocks. Thin sections from each block was stained with Masson trichrome stain and infarct size calculated both as a percentage of the area of ventricle involved and as the sum of the epicardial and the endocardial infarct circumference divided by the sum of the total LV epicardial and endocardial circumferences. Morphometric analysis of the stained sections quantified the extent of the fibrosis and capillary density.

Acute Myocardial Infarction Studies

An acute myocardial infarction (MI) in mice was used to investigate the effect of selected dHPG on heart function. produced by reversible ligation of the left coronary artery (LCA), as described (Rezai et al., 2005. Methods Mol Med 112:223-38). Briefly, CD-1 male mice (6 to 11 weeks of age and 34 to 39 mg) were anaesthesized with ketamine (112 mg/kg)/xylazine (18 mg/kg) IP) and 4% isofluorane, intubated, and ventilated. During the procedure, mice were maintained on 0.5% to 2% isoflurane. By means of a left lateral thoracotomy, the proximal LCA was temporarily ligated with 8-0 prolene at the level of the left atrial appendage. The LCA ligation was sustained for 30 minutes at which time it was released allowing for reperfusion. An alkylated C18 dHPG (IC-35) with metabolic effects in heart muscle cells was administered just before LCA ligation at a dose to achieve final circulating levels of 50 μM; this time point corresponds to the clinical setting of patients undergoing open heart surgery where such an agent might be given prior to surgery. A separate group of mice with an acute MI received an infusion of saline (the vehicle for dHPGs) and served as controls. The thoracotomy incision was closed and the animals came off the ventilator. Five days later the mice were re-anesthetized, as described above, for functional evaluation and euthanasia. Mice were euthanized under deep isoflurane anesthesia by injection of potassium chloride and removal of the heart.

Heart function in vivo was measured non-invasively in anesthetized mice, prior to thoracotomy on day 0 and day 5, by echocardiography using a Visual Sonics 700 VEVO system with a 30 MHz probe, probe holder and data analysis unit (Walinski et al., 2007 PNAS; Rottman et al., 2007. Echocardiography 24(1):83-9). Heart function, including systolic and diastolic left ventricular pressure (LVP), heart rate, and pressure-volume relationships, of the mice was also determined at 5 days, just prior to termination, by means of a microtip pressure transducer in the left ventricular cavity, placed there via the apex of the left ventricle (Joho et al., 2007. Am J Physiol Heart Circ. Physiol 292(1):H369-77; Pacher et al, 2008. Nature Protocols 3(9): 1422-1434).

### Example 1

### Alteration in Lactate Concentration by HPG Polymers in Heart Muscle Cells

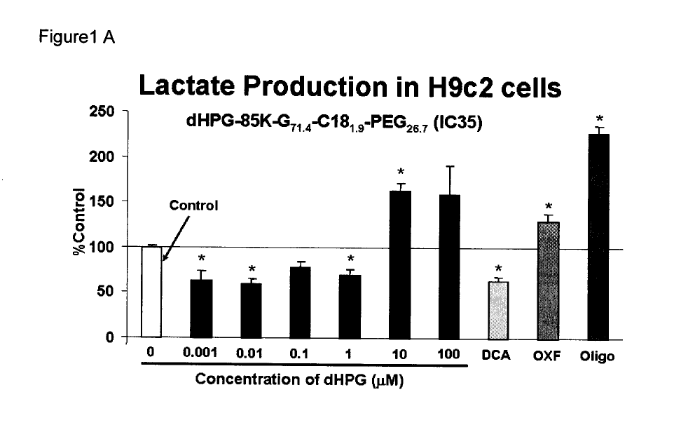

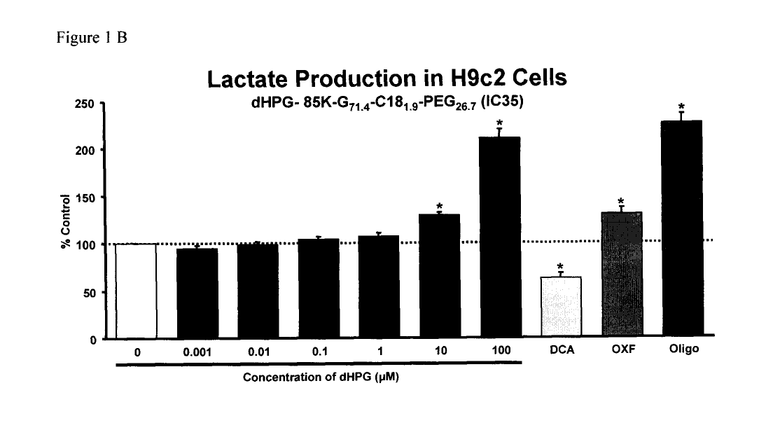

A variety of dHPG polymers were administered to H9C2 cells in culture and the lactate production analyzed (Tables 2, 3; FIGS. 1a, b). FIG. 1a shows an initial assay of lactate production; FIG. 1b and Tables 2, 3 show a repeat of the original lactate production in which a more accurate normalization procedure was used, providing greater consistence across repetition. Concentration range data (0.0001 to 250.0 uM), as well as controls (dichloroacetate-7.5 mM, DCA-BDH), a group exposed to oxfenicine (2 mM, OXF-Fluka), and a group exposed to oligomycin (2.0 μM, Oligo-Sigma). Exemplary results for HPG polymer IC35 (dHPG-39K-G₇₈-C18_{1.6}-PEG₂₀) are shown in FIG. 1.

Lactate concentration decreased with lower concentrations of dHPG polymer (0.001 to 1 uM) (FIG. 1b). An alkylated polymer concentration of 10 or 100 uM resulted in an increased concentration of lactate. Samples treated with DCA showed a reduction in lactate concentration. Samples treated with OXF showed an increase in lactate. Samples treated with oligo showed an increase in lactate concentration, greater than that of OXF-treated samples.

At higher concentrations (10, 100 uM) the effect observed is similar to that of OXF or oligo.

HPGs with alkyl (C18 or C10) chains increase lactate production at higher concentrations. This response may be independent of MW as demonstrated by RKK108, RKK259 and IC40 as compared to the other dHPGs with alkyl chains—Table 3).

HPGs containing C10 chains may not alter lactate production, even though they have been demonstrated to bind binding fatty acids (Table 3).

HPG core and PEG350 may not affect lactate production.

HPGs lacking alkyl chains (non-alkylated HPGs) may not alter lactate production at higher concentrations (above 1 μM). The same appears to be the case at lower concentrations as well.

TABLE 2
Polymers used in H9C2 and rat heart metabolic studies

| | | | | | | Palmitic acid | |
|---|---|---|---|---|---|---|---|
| | Experiment | | R | PEG | | binding | Mol. Wt |
| Polymers | Number | glycidol | mole % | mole % | Remarks | capacity(mol./mol) | (Mₙ) |
| dHPG-39K-G₇₃-C18₀-PEG₂₇ | RKK-99 | 73 | 0 | 27 | No alkyl groups | 0.0 | 39,000 |
| dHPG-85K-G_{71.4}-C18_{1.9}-PEG_{26.7} | RKK-108 | 71.4 | 1.9 | 26.7 | Higher MW | 0.55 | 85,000 |
| dHPG-80K-G₆₉-C18_{3.2}-PEG_{27.8} | RKK-153 | 69 | 3.2 | 27.8 | More hydrophobic compared to | 2.8 | 80,000 |
| | | | | | RKK108 | | |
| dHPG-36K-G₈₁-C18_{1.5}-PEG_{17.5} | RKK-108′, | 81 | 1.5 | 17.5 | | 0.3 | 36,000 |
| | IC-6 | | | | | | |
| dHPG-37K-G_{74.9}-C18_{1.4}-PEG_{16.2}- | RKK-108″ | 74.9 | 1.4 | 16.2 | 108′ modified with sulphonic | 0.3 | 37,000 |
| (SO₃H)_{7.5} | | | | | acid groups | | |
| dHPG-180K-G_{68.5}-C10₁₃-PEG_{18.5} | RKK-259 | 68.5 | 13 | 18.5 | C10 chains | 20.8 | 180,000 |
| dHPG-39K-G₇₈-C18_{1.6}-PEG₂₀ | IC-35 | 78 | 1.6 | 20 | Similar to RKK-108′, | 0.3 | 39,000 |
| dHPG-35K-G₆₉-R₀-PEG₃₁ | IC-72 | 69 | 0 | 31 | No alkyl groups, similar to | 0.0 | 35,000 |
| | | | | | RKK-99 | | |
| dHPG-91K-G_{75.8}-C18_{2.2}-PEG_{18.2}-N_{3.8} | IC-40(1) | 78 | 2.1 | 19.5 | Amine groups | 12 | 91,000 |
| dHPG-33K-G_{93.2}-C10_{3.4}-PEG₀-N_{3.4} | IC-70 | 93.2 | 3.4 | 0 | polyethyleneimine (PEI)core, | 8 | 33,000 |
| | | | | | HPG, high fatty acid binding | | |
| | | | | | capacity | | |
| dHPG-36.9K-G₇₅-R₀-PEG₂₅ | IC-214 | 75 | 0 | 25 | no alkyl groups, similar to | n.d. | 36900 |
| | | | | | RKK-99 | | |
| PEG-350 | PEG-350 | 0 | 0 | 100 | Methoxy poly(ethylene glycol) | 0.0 | 350 |
| HPG-25K | RKK-8 | 100 | 0 | 0 | Hyperbranched polyglycerol | 0.0 | 25,000 |

n.d.—no data

TABLE 3
Effect of dHPGs on Lactate Production by H9C2 Cells

| | 0.001 μM | 0.01 μM | 0.1 μM | 1.0 μM | 2.5 μM | 10.0 μM | 25.0 μM | 100.0 μM | 250.0 μM | |
|---|---|---|---|---|---|---|---|---|---|---|
| dHPG-85K-G_{71.4}-C18_{1.9}- | 88 | 92 | −29 | 103 | | 132 | | 185 ± 23 | | |
| PEG_{26.7} (RKK108) | | | | | | | | | | |
| dHPG-36K-G₈₁-C18_{1.5}- | 85 ± 9 | 100 ± 2 | 93 ± 6 | 89 ± 4 | | 118 ± 2 | | 164 ± 9* | | |
| PEG_{17.5} (RKK108′) | | | | | | | | | | |
| dHPG-36K-G₈₁-C18_{1.5}- | 115 ± 8 | 111 ± 4 | 107 ± 19 | 106 ± 2 | | 166 ± 6* | | 180 ± 12* | | |
| PEG_{17.5} (IC6) | | | | | | | | | | |
| dHPG-39K-G₇₈-C18_{1.6}- | 95 ± 3 | 99 ± 3 | 104 ± 3 | 107 ± 4 | | 129 ± 3* | | 211 ± 9* | | |
| PEG₂₀ (IC35) | | | | | | | | | | |
| dHPG-91K-G_{75.8}-C18_{2.2}- | 92 ± 5 | 101 ± 2 | 104 ± 8 | 97 ± 5 | | 98 ± 5 | | 123 ± 3* | | |
| PEG_{18.2}-N_{3.8}(IC40(1)) | | | | | | | | | | |
| dHPG-33K-G_{93.2}-C10_{3.4}- | 105 ± 5 | 110 ± 4 | 115 ± 3* | 108 ± 8 | | 115 ± 4* | | 157 ± 4* | | |
| PEG₀-N_{3.4} (IC70) | | | | | | | | | | |
| dHPG-180K-G_{68.5}-C10₁₃- | 103 ± 5 | 104 ± 4 | 111 ± 4 | 116 ± 5* | | 199 ± 12* | | | | |
| PEG_{18.5} (RKK259) | | | | | | | | | | |
| dHPG-39K-G₇₃-C18₀- | | | | | 108 ± 7 | | 104 ± 5 | | 116 ± 10 | |
| PEG₂₇ (RKK99) | | | | | | | | | | |
| dHPG-35K-G₆₉-C18₀- | 102 ± 4 | 102 ± 4 | 113 ± 4* | 105 ± 6 | | 111 ± 4* | | 103 ± 4 | | |
| PEG₃₁ (IC72) | | | | | | | | | | |
| dHPG-36.9K-G₇₅-C18₀- | | | | | | 102 ± 4 | | 101 ± 7 | | |
| PEG₂₅ (IC-214) | | | | | | | | | | |
| dHPG-25K-G₁₀₀ (RKK8) | 101 ± 2 | 102 ± 1 | 105 ± 5 | 98 ± 3 | | 99 ± 5 | | 106 ± 4 | | |
| PEG350 | 93 ± 6 | 99 ± 4 | 110 ± 3 | 91 ± 7 | | 105 ± 5 | | 113 ± 9 | | |
| DCA | | | | | | | | | | 63 ± 5* |
| Oxfenicine | | | | | | | | | | 130 ± 7* |
| Oligomycin | | | | | | | | | | 226 ± 10* |

Data are expressed as Mean ± SEM and as % Control with Control being equal to 100%.
Concentrations of DCA (dichloroacetate), oxfenecine, and oligomycin used were 7.5 mM, 2 mM, and 2 μM, respectively.
N = 2 to 26 per group.
dHPG, derivatized hyperbranched polyglycerol.
G, glycerol.
C, alkyl chains of 18 or 10 carbons when present.
PEG, poly(ethylene glycol) of molecular weight 350.
N, amine group.
Average molecular weight is indicated by the second term. K, × 10³.
Subscript numbers represent constituent molar fraction as a percent.
dHPG preparation lot number is indicated in parenthese

### Example 2

### Function of Isolated Working Hearts

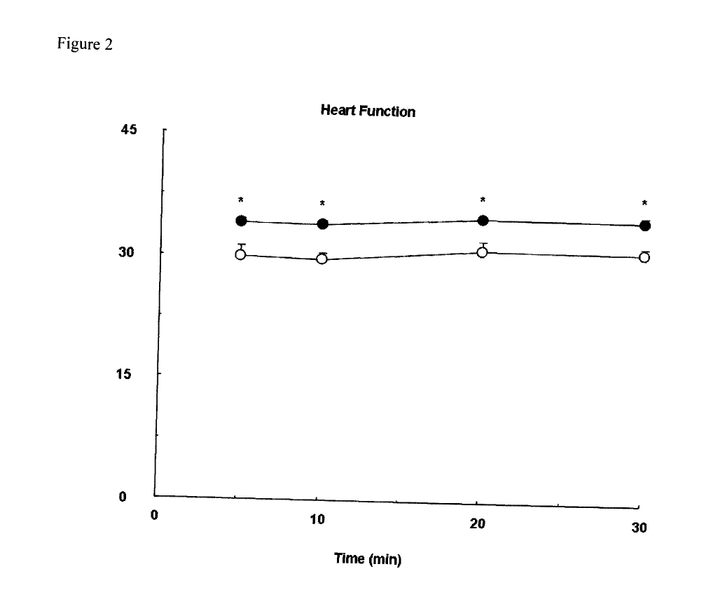

Isolated, working rat hearts were exposed to 10 μM dHPG-85K-G_{71.4}-C18_{1.9}-PEG_{26.7}(RKK-108) and studied under normoxic, non-ischemic conditions. During normoxic, non-ischemic perfusion, RKK108 improves heart function (rate-pressure assessment—beats per minute×mmHg/1000) by about 10 to about 15% (FIG. 2), When heart function is assessed by hydraulic work an improvement of about 20% is also observed. A similar experiment, treating hearts with oxfenicine at 2 mM concentration showed a similar trend.

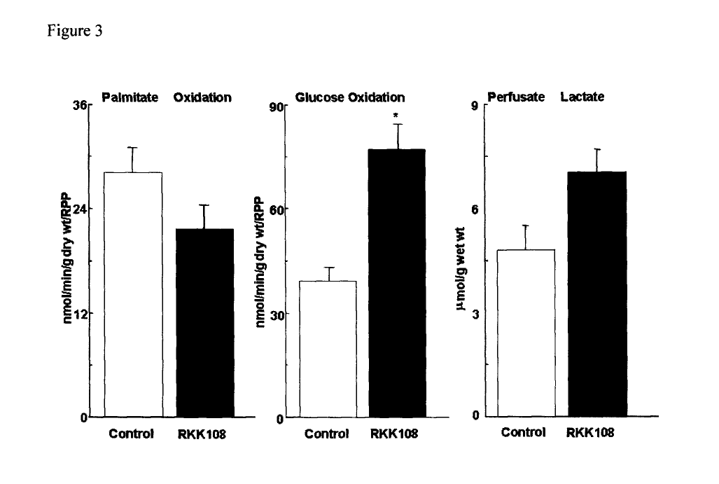

RKK108 reduces palmitate oxidation and stimulates both glucose oxidation and accumulation of lactate in the perfusate (FIG. 3). Elevation in lactate is a reflection of an increased rate of glycolysis. This increased accumulation of lactate is similar to that seen with the same concentration of RKK108 (1 mg/ml, or 11.8 uM) administered to H9C2 cells. The results obtained with RKK108 (FIG. 3) are similar to those seen in hearts exposed to oxfenicine, an inhibitor of fatty acid oxidation, that reduces palmitate oxidation and accelerates both glucose oxidation and glycolysis.

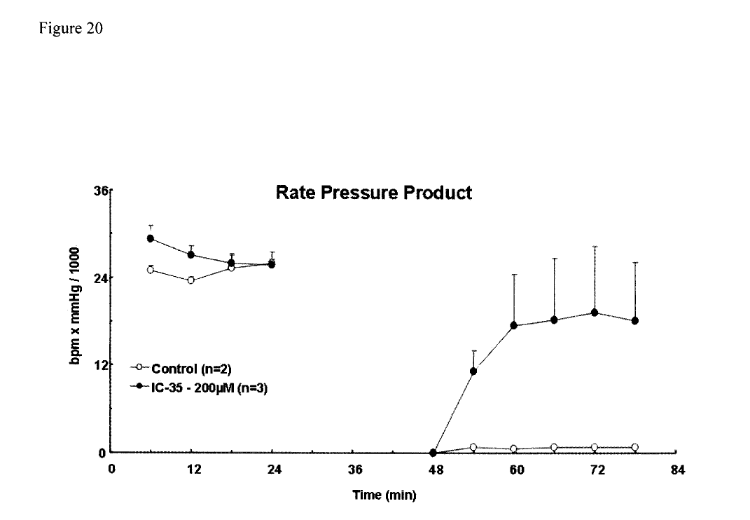

Administration of dHPG (IC35 polymer) demonstrated an improvement in heart function following ischemic stress (FIG. 20).

dHPGs have metabolic and functional effects on intact, working hearts, similar to those produced by a known myocardial metabolic modulator, oxfenicine.

Additionally, the correspondence of findings in isolated hearts and H9C2 cells indicates that metabolic effects of dHPGs in H9C2 cells may be extrapolated to hearts.

### Example 3

### Metabolism and Function of Ischemic Isolated Working Hearts

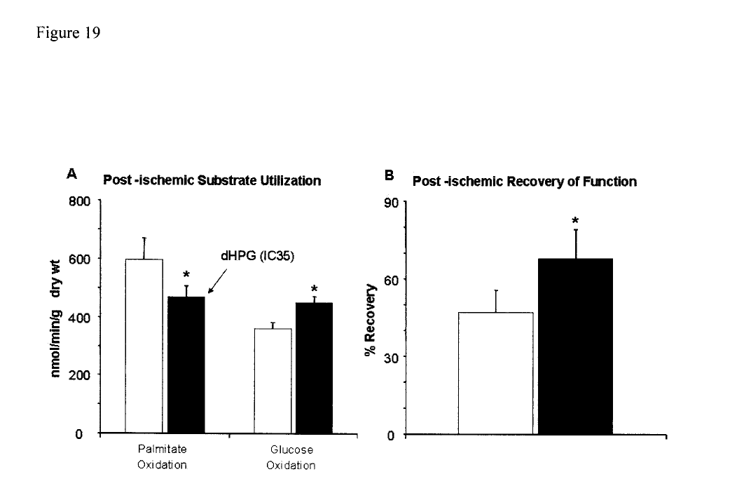

FIG. 19 shows the effect of C18 dHPG (IC35, dHPG-39K-G₇₈-C18_{1.6}-PEG₂₀) at 20 to 50 μM on substrate utilization (A) and recovery of function (B) during reperfusion after 24 min of no-flow global ischemia in isolated working rat hearts perfused with 1.2 mM [9,10-³H]-palmitate, 5.5 mM [U-¹⁴C]-glucose, 0.5 mM lactate, and 20 mU/l insulin. Concentrations of insulin and substrates reflect values seen in physiological and pathophysiological conditions; the concentration of palmitate recapitulates that seen during myocardial ischemia. Alkylated C18 dHPG (IC-35) at 200 μM had a dramatic effect on recovery of function, resulting in nearly 80% recovery of pre-ischemic function (FIG. 20).

Alkylated dHPG (IC-35) improves post-ischemic functional recovery as compared to controls (FIG. 19, 20, 23). Non-alkylated dHPG (IC-72, IC-214) may also demonstrate a beneficial effect on functional recovery of the heart following ischemia and reperfusion.

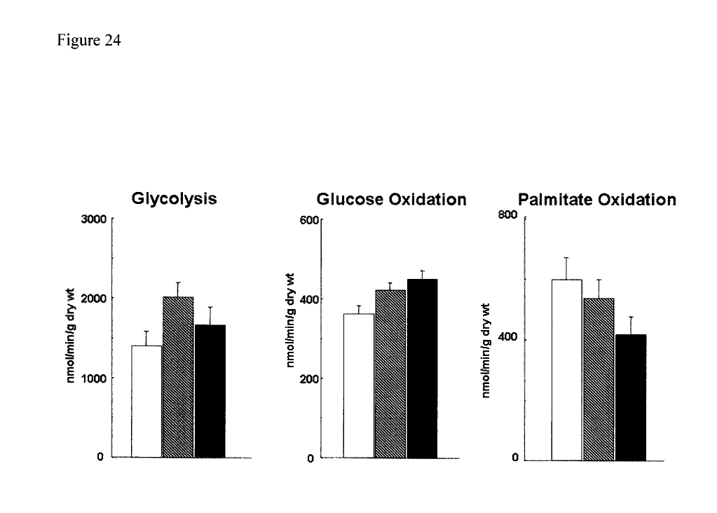

In an isolated working heart, the effect of 20 micromolar alkylated (IC-35) and non-alkylated (IC-72) dHPG on glycolysis, glucose oxidation and palmitate oxidation is illustrated (FIG. 24). Some increase in glycolysis is effected by IC72 and both dHPGs increase glucose oxidation relative to controls, although the effect is greater for IC35. IC-35 reduces palmitate oxidation relative to control, a reduction in palmitate oxidation superior to that with IC-72.

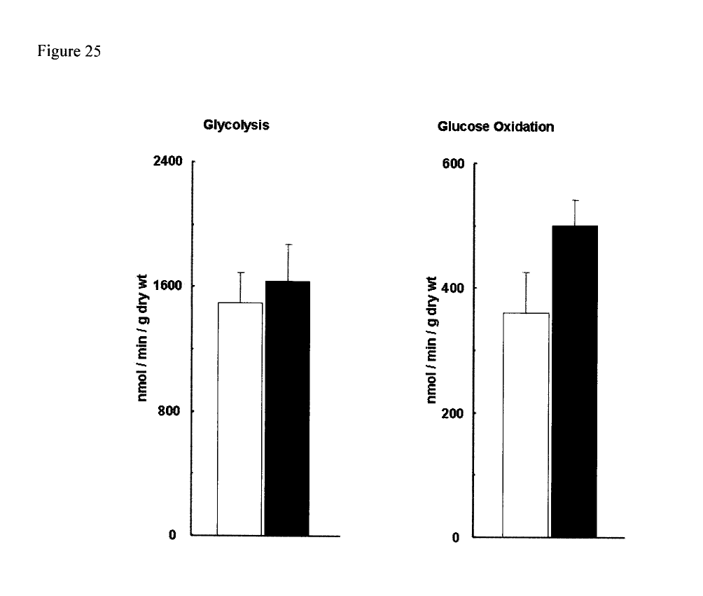

FIG. 25 shows an effect of 50 micromolar IC-35 on substrate use (glycolysis and glucose oxidation) in isolated working rat hearts after ischemia. Compared to the data presented in FIG. 24, increasing the concentration of polymer demonstrated an increase in stimulation of glucose oxidation.

### Example 4

### Isovolemic Blood Exchange Studies

The perfusion of polymer dHPG-36K-G₈₁-C_{181.5}-PEG_{17.5} (RKK108′) had minimal effects on the cardiovascular system of the animals that received the 15% total blood volume exchange (TVE) of polymer. The mean HR (335 BPM) and PP (mean of systolic/diastolic) were relatively stable and constant for the majority of the post-infusion period suggesting the polymer had little or no major effect on the ability of the heart to regulate contractility, heart rate and blood pressure at this dose. The mean PP of 77 mm Hg (approx. 96/57) in the post-infused animal displayed little fluctuations except for when the anesthetic was getting light, such as the final 30 minutes before termination.

FIG. 4a illustrates the effects of the polymer on blood pH, as well as the effects of the polymer vehicle, either Ringer;s lactate (“Ringer's”) or NaCl saline. The results show that the high dose 1.2 mM polymer in saline has a significant inhibitory effect on blood pH. This drop in pH is consistent with the poorer buffering capacity of saline in circulating blood and the preferred use of lactated Ringers as described (Williams et al 1999. Anesth Analg 88:999-1003).

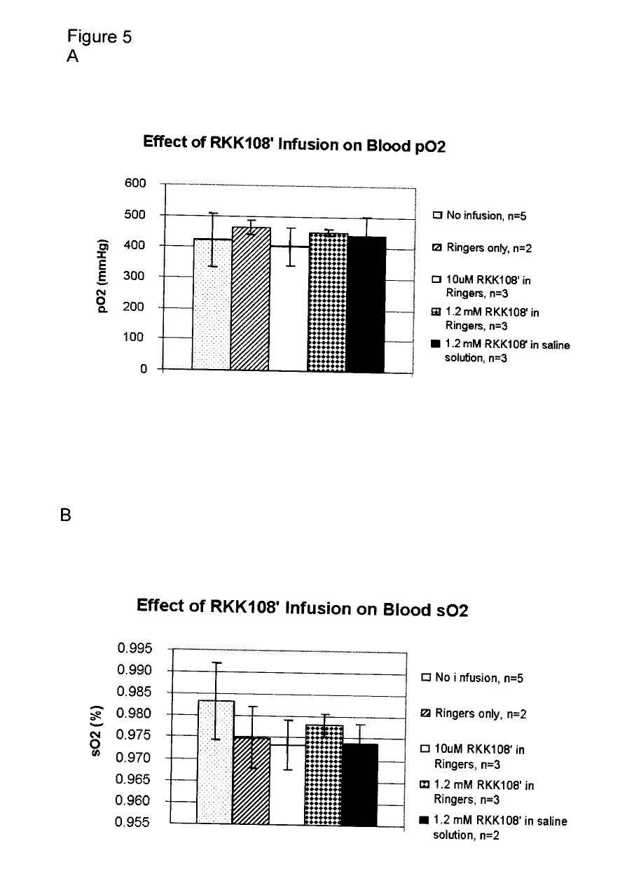

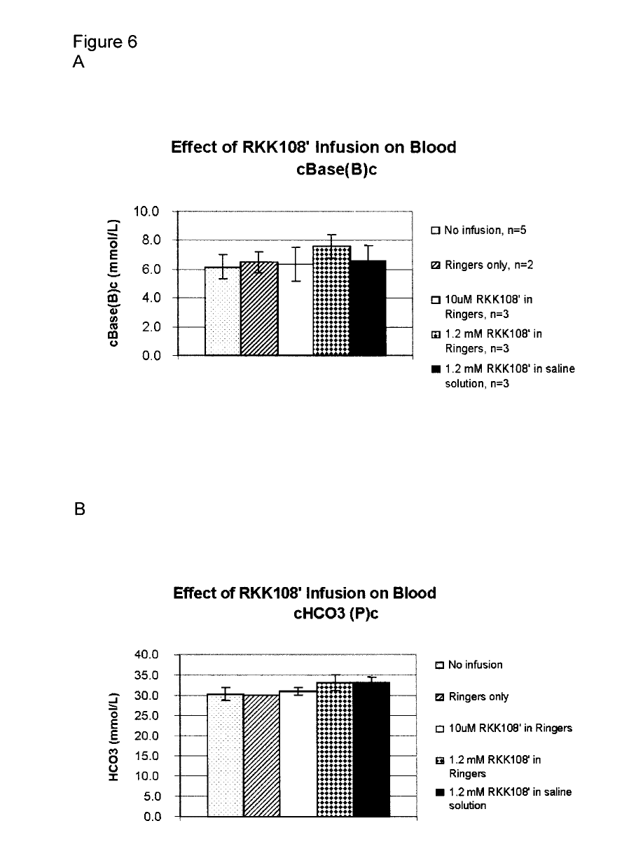

Blood gases demonstrate that the polymer has no disconcerting effects on blood chemistry. A marginal elevated effect from the polymer in saline on the blood pCO₂ levels is observed, which reflects alveolar ventilation relative to the metabolic rate (FIG. 4b). Neither arterial oxygen tension (pO₂) or the oxygen saturation (sO₂) (reflecting the % of oxygenated hemoglobin in relation to the amount available) were affected by polymer (FIGS. 5a,b). The ability of the blood to buffer itself against changes in pH was also unaffected by infusion of the polymer as indicated by the lack of changes in cHCO₃ (bicarbonate) and cBase. All observed values in the polymer treated animals were within the normal range of values in the control animals (FIGS. 6a, b).

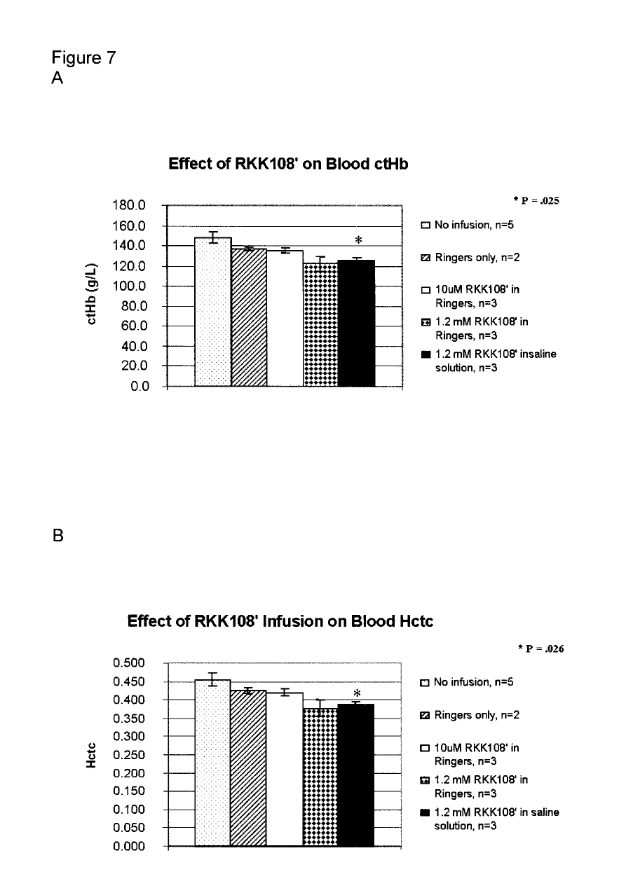

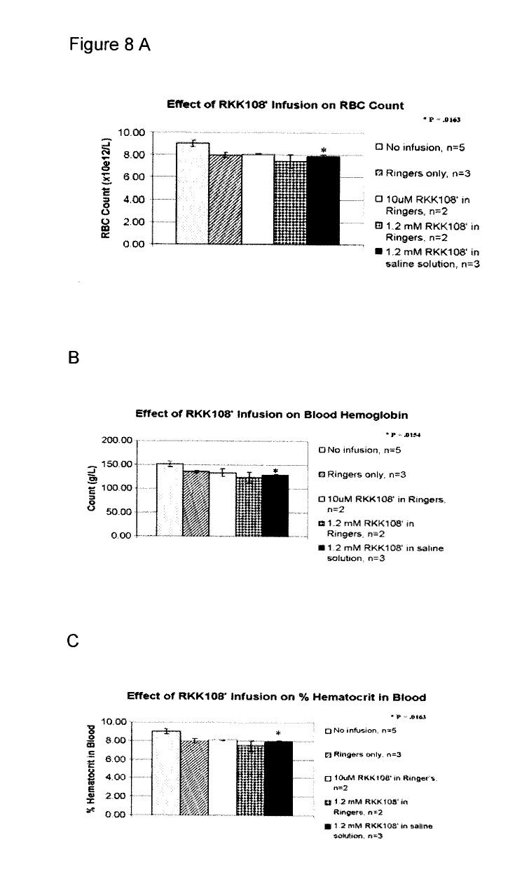

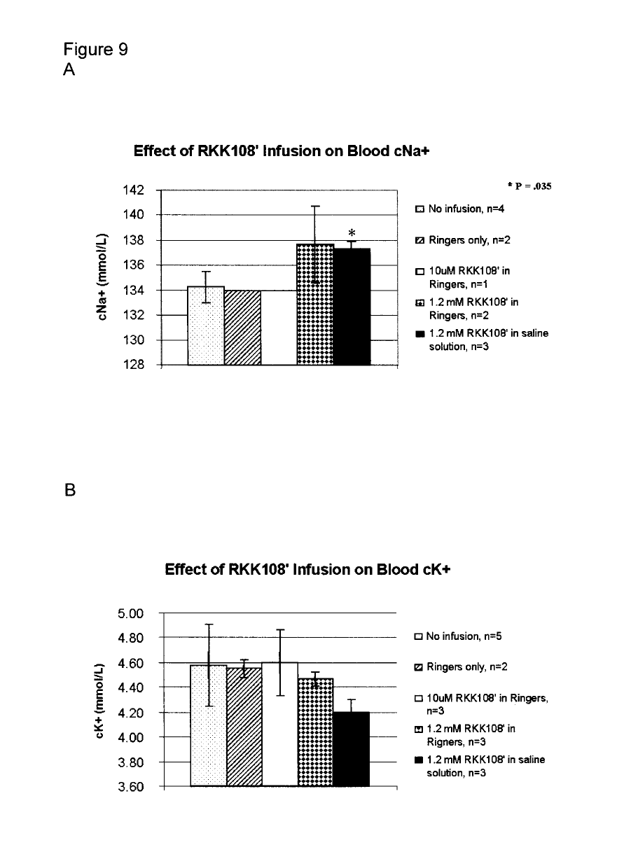

1.2 mM polymer in saline buffer decreased the total blood hemoglobin and hematocrit (FIGS. 7a, b), consistent with the observed reduction in RBC count, blood hemoglobin, and % hematocrit (FIGS. 8a, b, c). Only animals treated with high dose polymer in saline showed significantly altered blood electrolytes (cNa⁺, cCa²⁺cK⁺, cCl⁻), specifically, elevated cNa⁺ and cCa²⁺ levels (FIGS. 9a, b and 10a, b).

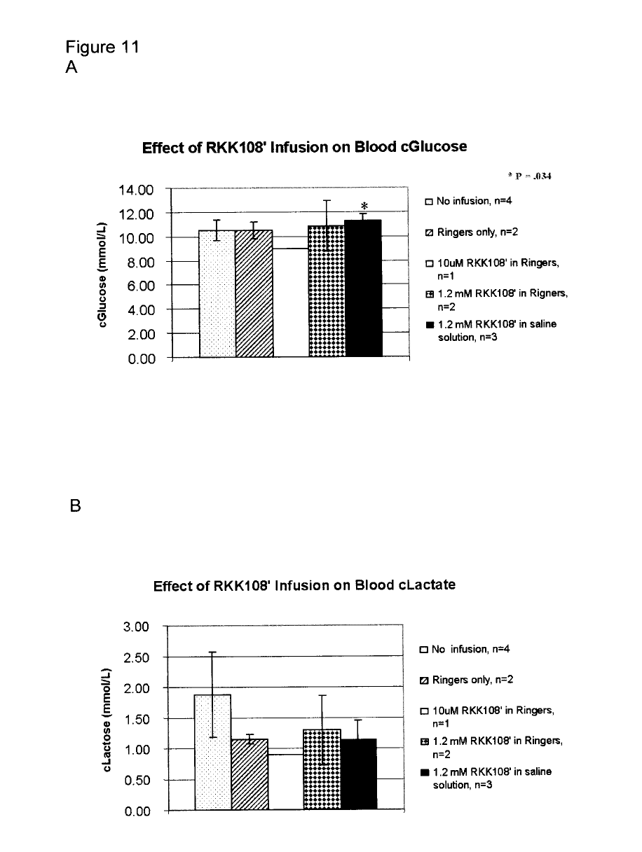

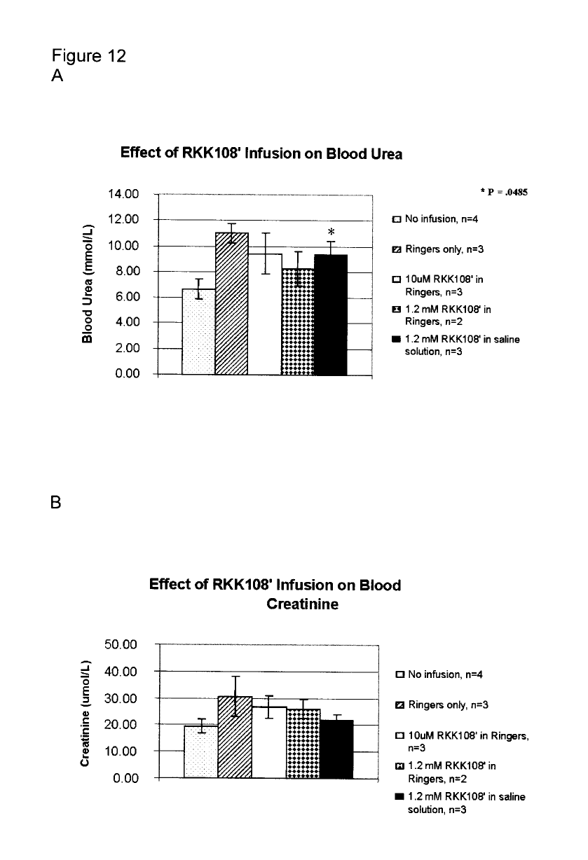

The effects of polymer on metabolism was measured, with glucose levels higher only in the animals treated with 1.2 mM doses of polymer in saline and so any positive metabolic effect that could be measured systemically would be reflective of an action of the polymer at sites other than just the heart (FIG. 11a). There was no effect of polymer on the lactate levels of any animal treatments (FIG. 11b). Blood urea was elevated in the high dose polymer/saline treated animals (FIG. 12a), but there were no significant changes in creatinine levels (FIG. 12b).

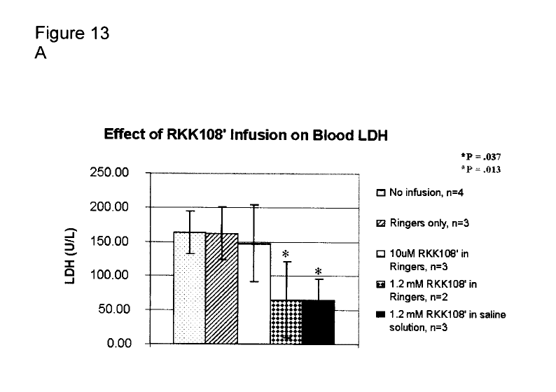

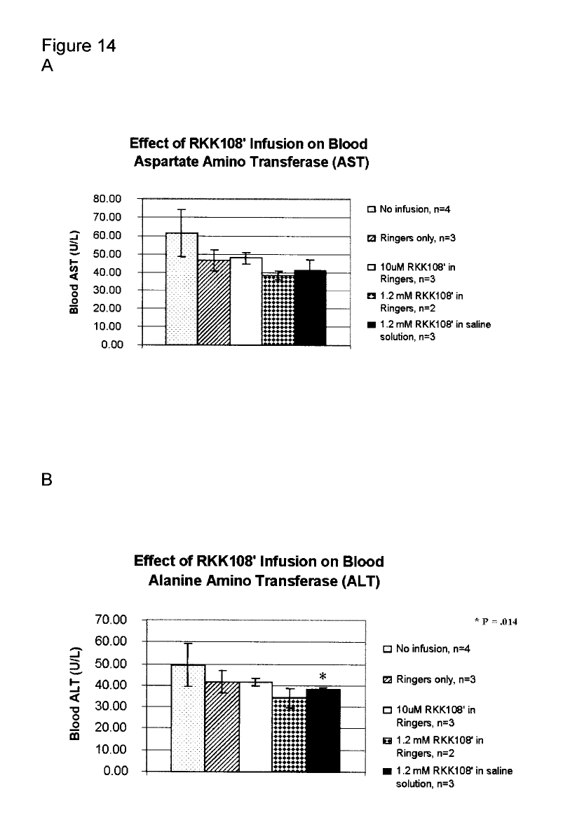

LDH, or lactate dehydrogenase is a measure of tissue injury, and its levels in the blood decrease with 1.2 mM RKK-108′ in both Ringers and saline solutions (FIG. 13a). RKK-108′ infusion did not affect AST liver enzyme levels, consistent with minimal levels of cell necrosis (FIG. 14a). A decrease in levels of the liver enzyme ALT with the 1.2 mM dose of RKK-108′ in Ringers' solution was observed (FIG. 14b).

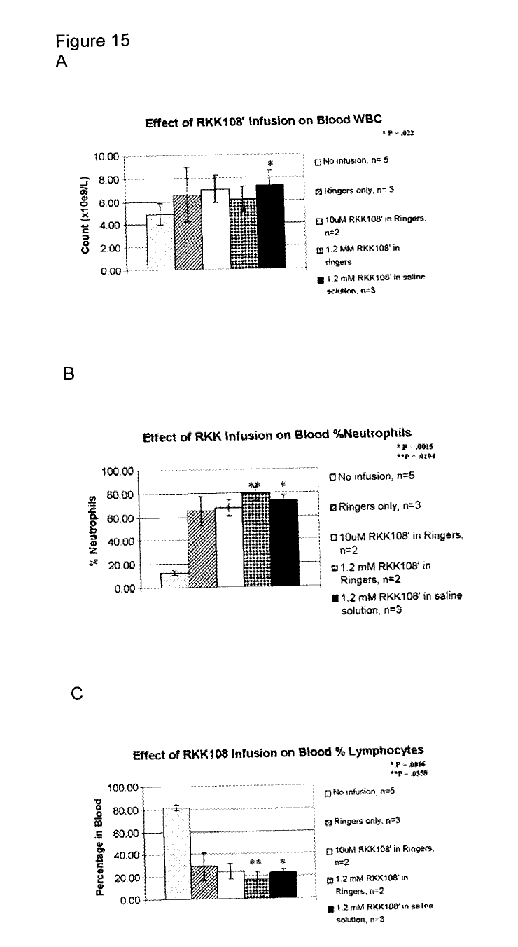

Minor changes are observed in blood total WBC counts FIG. 15a, with a significant change in total WBC count in 1.2 mM RKK-108′ in Ringer's solution. This may reflect the increase in neutrophils (% N) observed in animals treated with the higher dose of RKK-108′ (FIG. 15b). A decrease in the percentage of lymphocytes (% L) in the polymer treated animals was also observed (FIG. 15c).

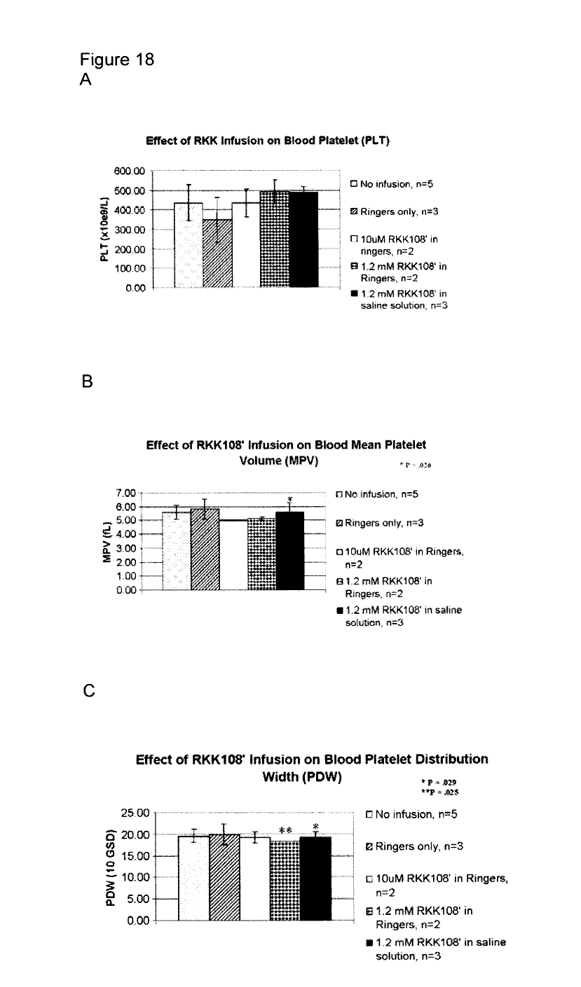

The RBC count and other measures of red blood cell parameters (% hematocrit, haemoglobin, MCV, MCH, MCHC, RDW) show an inhibitory effect of the polymer at the 1.2 mM concentration. This is consistent with blood loss and turnover of new RBC precursor cells (FIGS. 9a, b, c, 16a, b, 17a, b). High dose polymer in saline also had a small effect on platelets, with increases in mean platelet volume and platelet distribution width, but the increase is within a normal range (FIGS. 18a, b, c).

The polymer may be infused into animals without significantly altering the exchange of blood gases or blood cell numbers or functions, or inducing indicators of tissue injury. Some elevated values observed may be associated with differences in response to surgical insult compared to the controls.

### Example 5

### Effects of dHPG on Heterotopic Heart Transplants

The gross appearance of both hearts treated with polymer prior to transplant (N=2) were bright pink and flushed with blood on the epicardial surface and generally healthier-looking than the saline/heparin treated hearts of control rats, which had many areas of distinct focal necrosis. Microscopic analysis of trichrome and H&E stained heart tissues from the donor hearts treated with an alkylated hyperbranched polyglycerol RKK108″ for approximately 20 min prior to transplantation had a pronounced effected preserving the morphology of the heart and inhibiting the deposition of collagen in donor hearts.

The dHPG polymer inhibited development of interstitial fibrosis when used prior to immediate transplantation.

### Example 6

### Effect of Derivatized Hyperbranched Polyglycerols (dHPG) on Heart Function after Acute Myocardial Infarction in Mice

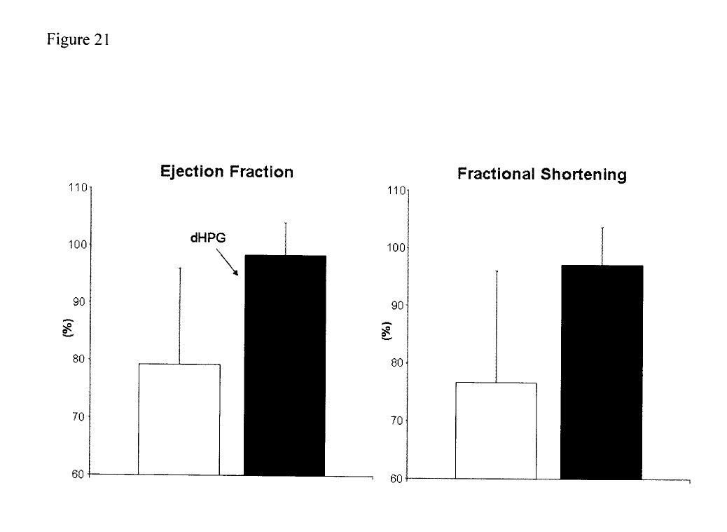

FIG. 21 shows the effect of dHPG (IC35) on post-ischemic function in vivo. Heart function was assessed non-invasively by echocardiography in mice administered saline or IC-35 just prior to ischemia. Data were obtained in anesthetized mice prior to thoracotomy at day 0 and day 5. Values are expressed as % of pre-ischemic function. N=3 per group.

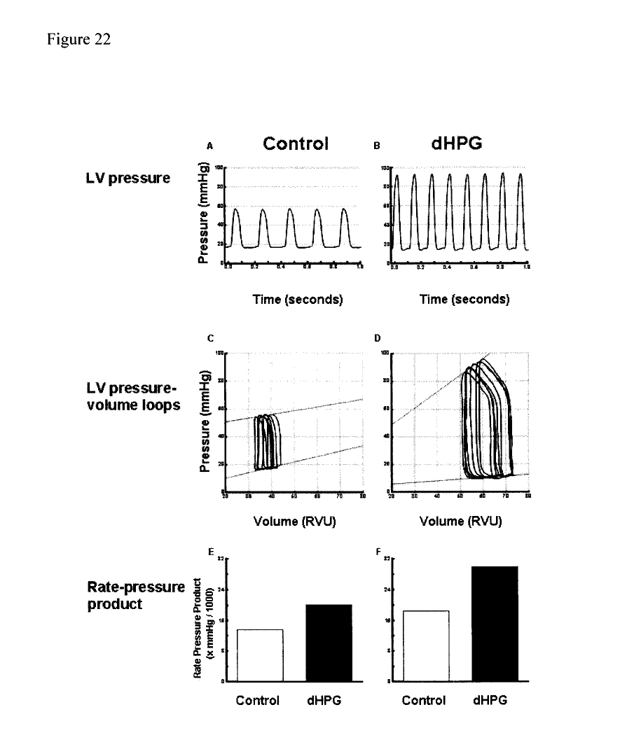

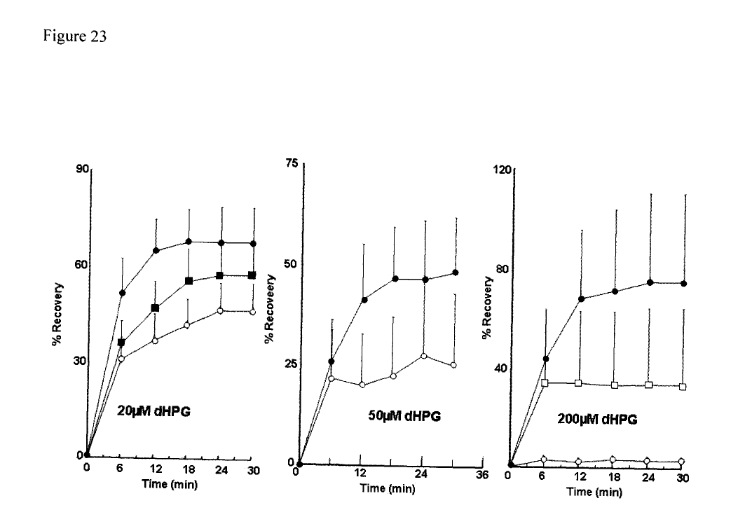

FIG. 22 shows the effect of dHPG on post-ischemic heart function in vivo. Left ventricular (LV) pressure signals were greater in the mice treated with the dHPG relative to control. LV pressure-volume loops in the dHPG treated animals were also greater, relative to control, indicating a superior left ventricular function. The heart rate-pressure product, a measure of external work, of hearts treated with dHPG was superior when administered either prior to ischemia (left graph) or upon reperfusion (right graph), relative to control.

While specific embodiments of the invention have been described and illustrated, such embodiments should be considered illustrative of the invention only and not as limiting the invention.

All citations are herein incorporated by reference.

One or more currently preferred embodiments have been described by way of example. It will be apparent to persons skilled in the art that a number of variations and modifications can be made without departing from the scope of the invention as defined in the claims.

What is claimed is:

1. A method of improving heart function in a subject, the method comprising administering an effective amount of a hyperbranched polyglycerol to a subject.

2. The method of claim 1, wherein improving heart function comprises one or more of an increase in myocardial contractile function, reduced or absent fibrosis, an increase in mechanical efficiency of the heart, an increase in ejection fraction, an increase in glucose oxidation or a decrease in fatty acid oxidation.

3. The method of claim 1 wherein the hyperbranched polyglycerol is alkylated.

4. The method of claim 3 wherein the alkylated hyperbranched polyglycerol is selected from the group consisting of RKK-43, RKK-55, RKK-56, RKK-71, RKK-108, RKK-108′, RKK-108″, RKK-259, IC35, IC70 and IC40(1).

5. The method of claim 1, wherein the hyperbranched polyglycerol is non-alkylated.

6. The method of claim 5 wherein the non-alkylated hyperbranched polyglycerol is selected from the group consisting of RKK-1, RKK-2, RKK-5, RKK-6, RKK-7, RKK-8, RKK-11, RKK-12, RKK-99, RKK-111, IC214 and IC72.

7. The method of claim 1 wherein the effective amount provides a concentration from about 0.001 μM to about 1000 μM in the blood.

8. The method of claim 3 wherein an alkyl chain of the alkylated hyperbranched polyglycerol is a 10-carbon alkyl chain (C10) or an 18-carbon alkyl chain (C18).

9. The method of claim 7 wherein the provided concentration is from about 20 μM to about 200 μM in the blood.

10. A pharmaceutical composition comprising a hyperbranched polyglycerol and a pharmaceutically acceptable carrier in an amount effective to improve heart function.

11. The pharmaceutical composition of claim 10, wherein improving heart function comprises one or more of an increase in myocardial contractile function, reduced or absent fibrosis, an increase in mechanical efficiency of the heart, an increase in ejection fraction, an increase in glucose oxidation or a decrease in fatty acid oxidation.

12. The pharmaceutical composition of claim 10 wherein the hyperbranched plyglycerol is alkylated.

13. The pharmaceutical composition of claim 12 wherein the alkylated hyperbranched polyglycerol is selected from the group consisting of RKK-43, RKK-55, RKK-56, RKK-71, RKK-108, RKK-108′, RKK-108″, RKK-259, IC35, IC70 and IC40(1).

14. The pharmaceutical composition of claim 10, wherein the hyperbranched polyglycerol is non-alkylated.

15. The pharmaceutical composition of claim 14 wherein the non-alkylated hyperbranched polyglycerol is selected from the group consisting of RKK-1, RKK-2, RKK-5, RKK-6, RKK-7, RKK-8, RKK-11, RKK-12, RKK-99, RKK-111, IC214 and IC72.

16. The pharmaceutical composition of claim 10 wherein the effective amount provides a concentration from about 0.001 μM to about 1000 μM in the blood.

17. The pharmaceutical composition of claim 12 wherein an alkyl chain of the alkylated hyperbranched polyglycerol is a 10-carbon alkyl chain (C10) or an 18-carbon alkyl chain (C18).

18. The pharmaceutical composition of claim 16 wherein the provided concentration is from about 20 μM to about 200 μM in the blood.
